(19) United States
(12) Patent Application Publication
(10) Pub. No.: US 2004/0062802 A1
(43) Pub. Date: Apr. 1, 2004

(54) Maximizing effectiveness of substances used to improve health and well being

(76) Inventor: Victor M. Hermelin, Chesterfield, MI (US)

Correspondence Address: Gary M. Nath, NATH & ASSOCIATES PLLC, 6th Floor, 1030 15th Street, N.W., Washington, DC, 20005, US

(21) Appl. No.: 10/644,041

(22) Filed: Aug. 20, 2003

Related U.S. Application Data
(63) Continuation of application No. 09/475,992, filed on Dec. 30, 1999.
Application No. 09/475,992 is a continuation-in-part of application No. 09/323,158, filed on Jun. 1, 1999, now Pat. No. 6,214,379.
Application No. 09/323,158 is a continuation of application No. 09/053,487, filed on Apr. 2, 1998, now Pat. No. 5,945,123.

(51) Int. Cl. A61K 9/22

(52) U.S. Cl. 424/468

(57) ABSTRACT

The present disclosure relates to novel dosage forms, drug delivery regimens, methods and pharmaceutical compositions which optimize the therapeutic effects of active therapeutic substances through the application of the concept of uneven dosing.

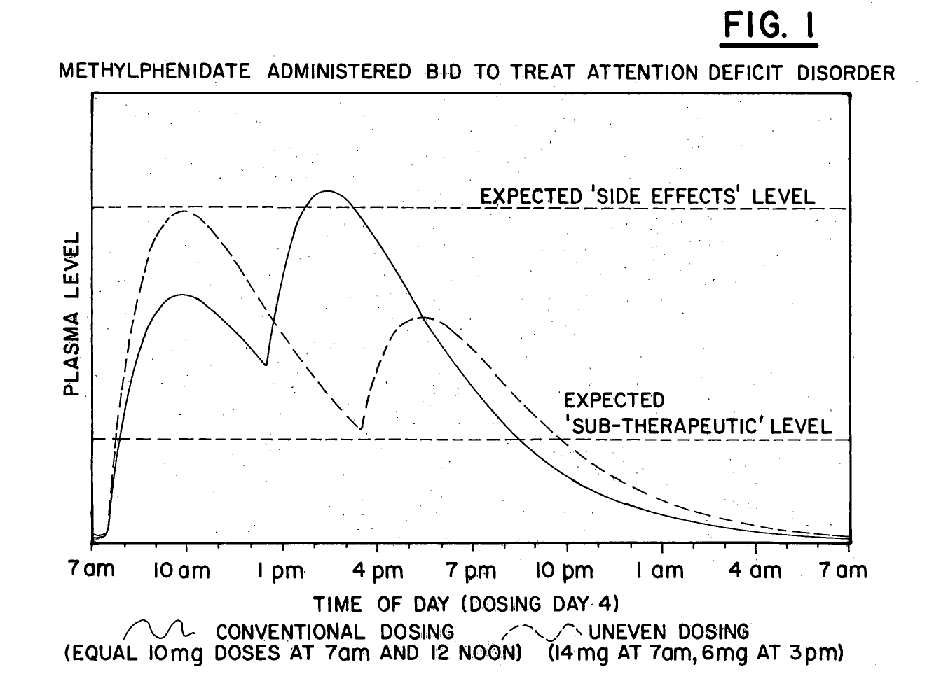

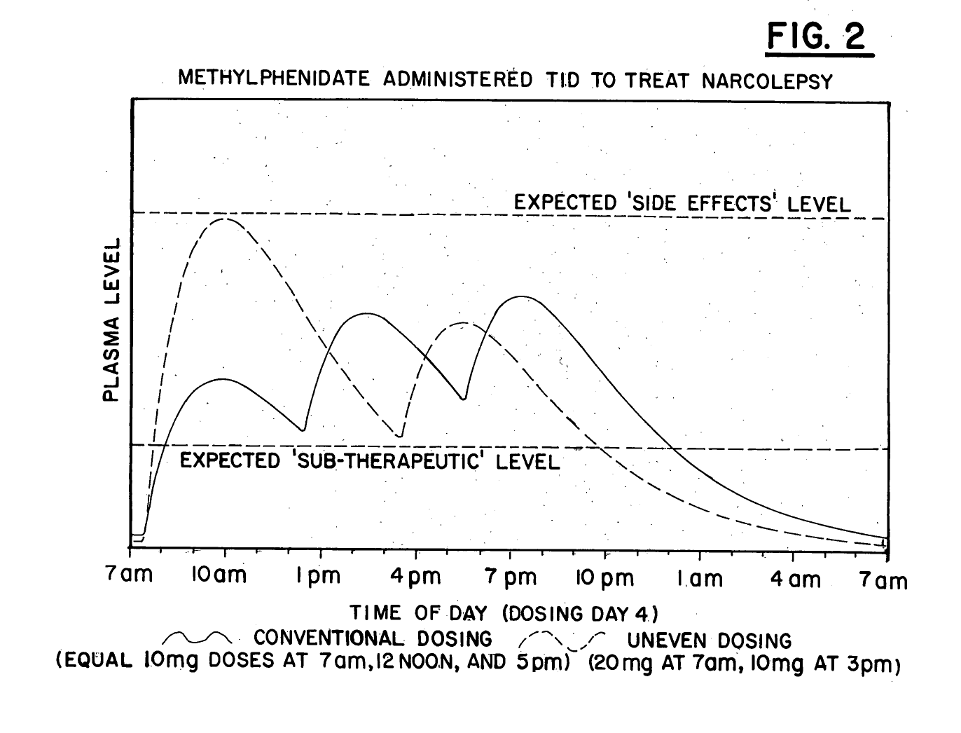

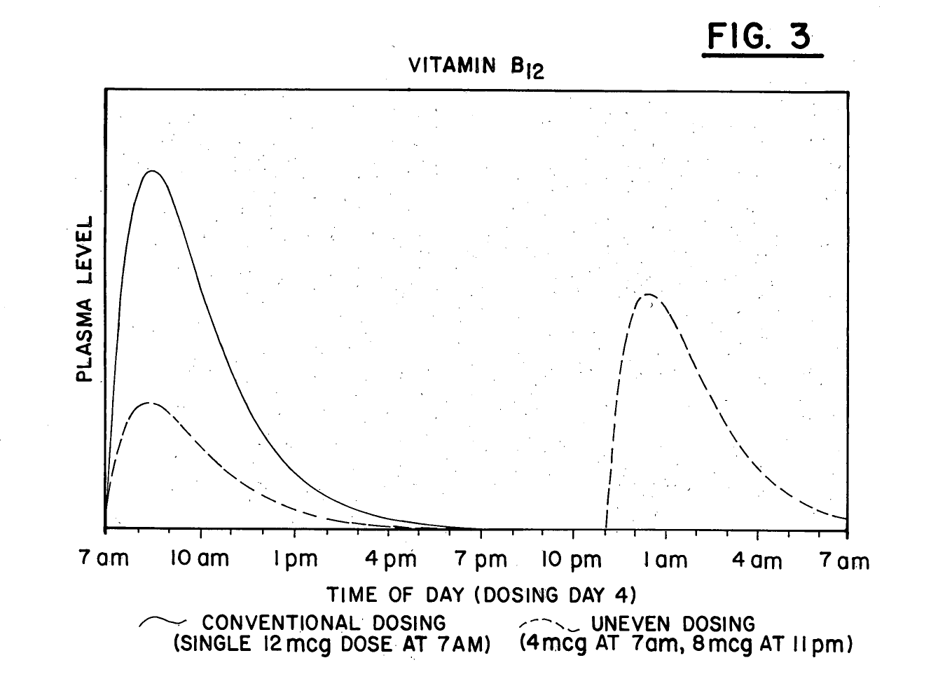

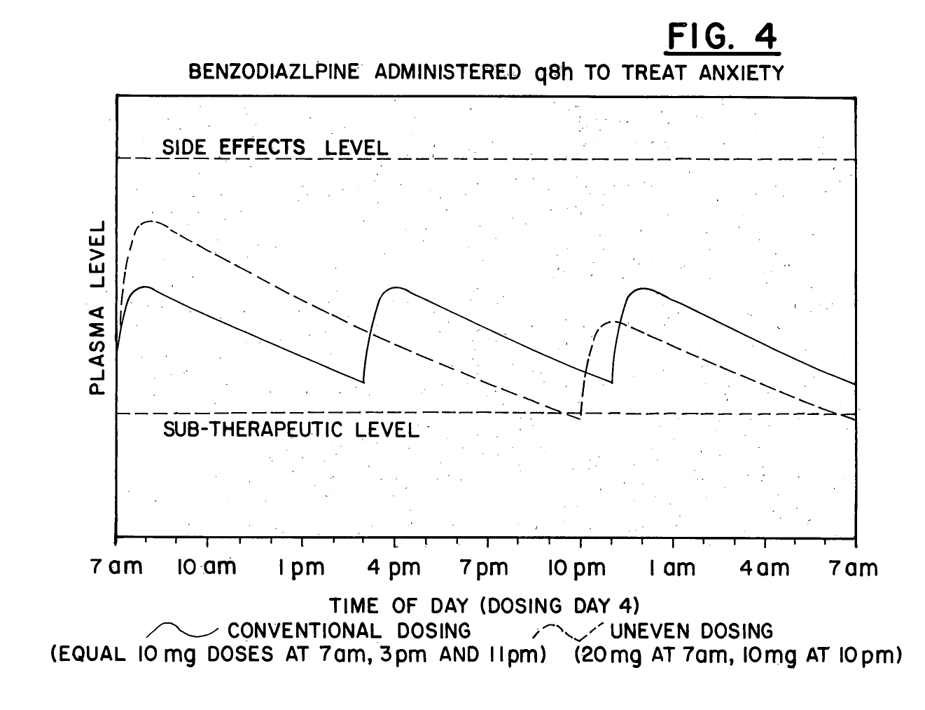

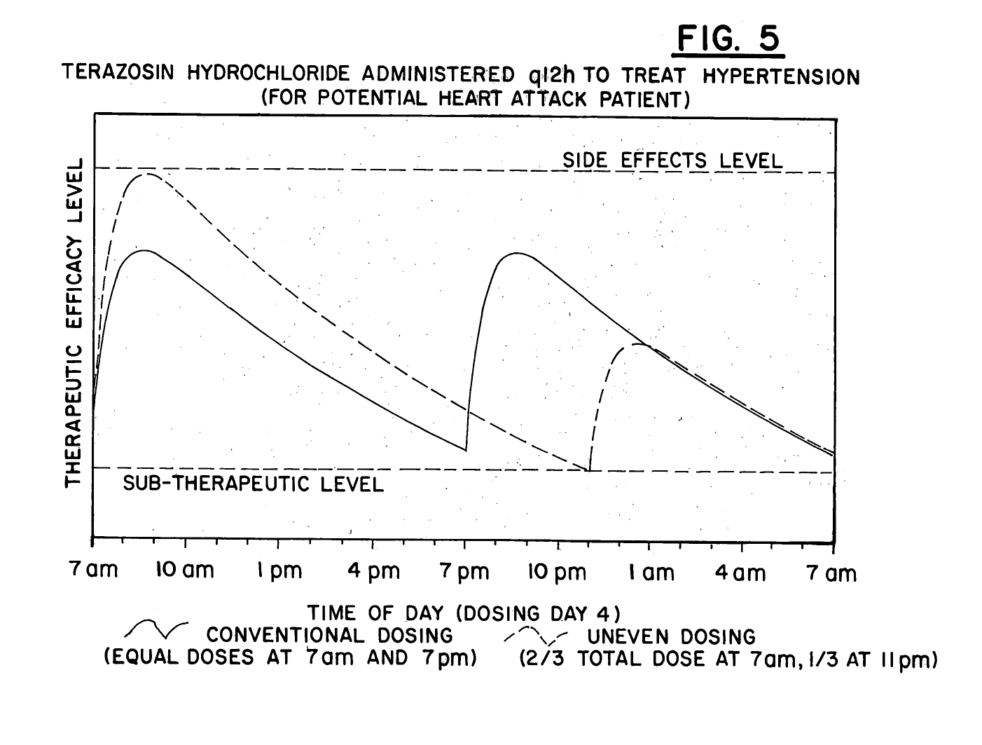

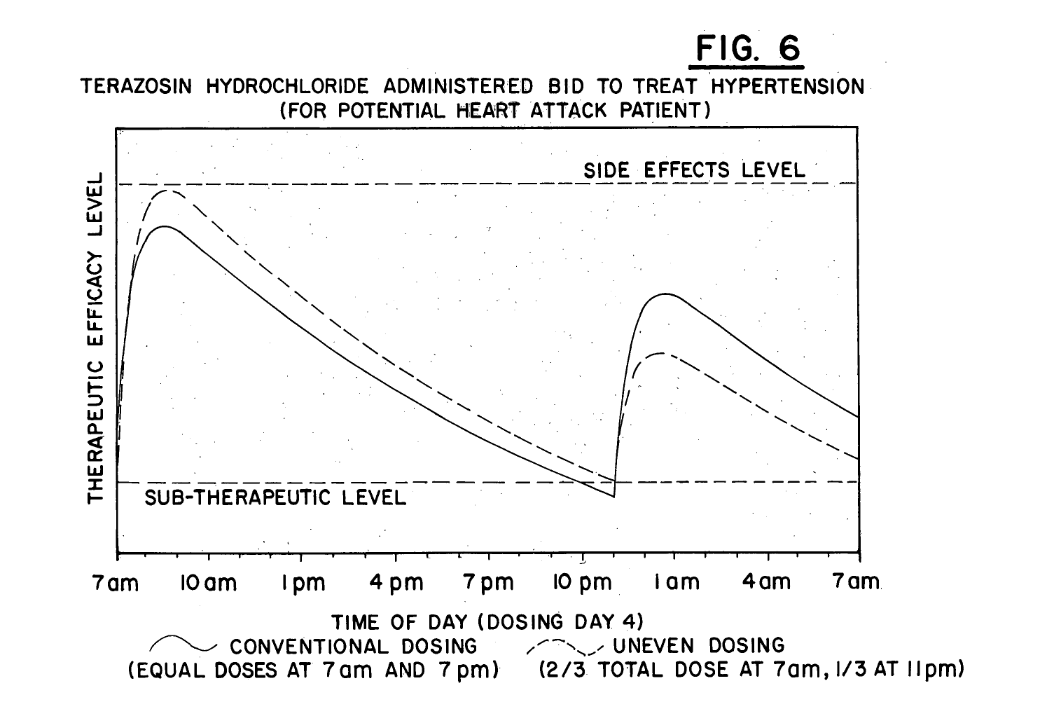

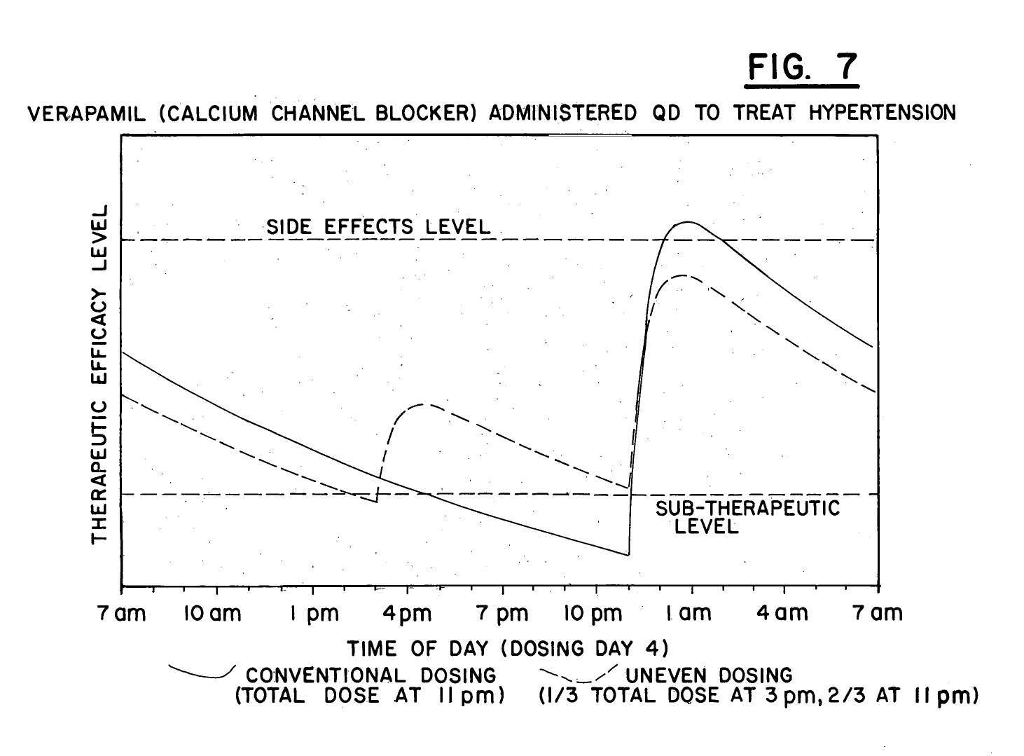

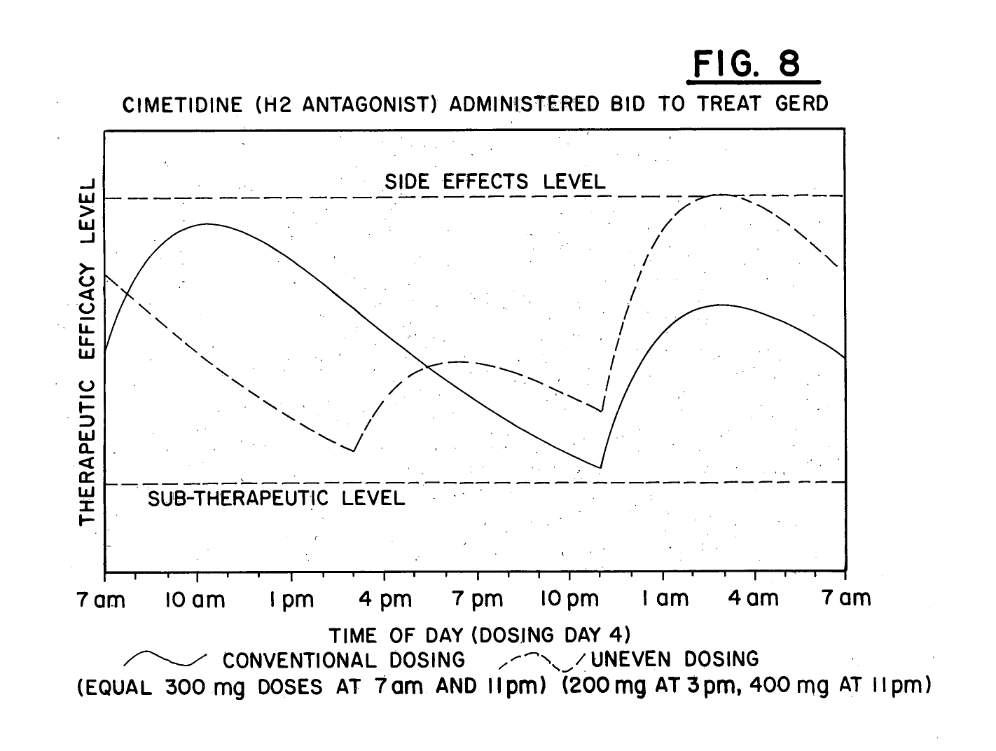

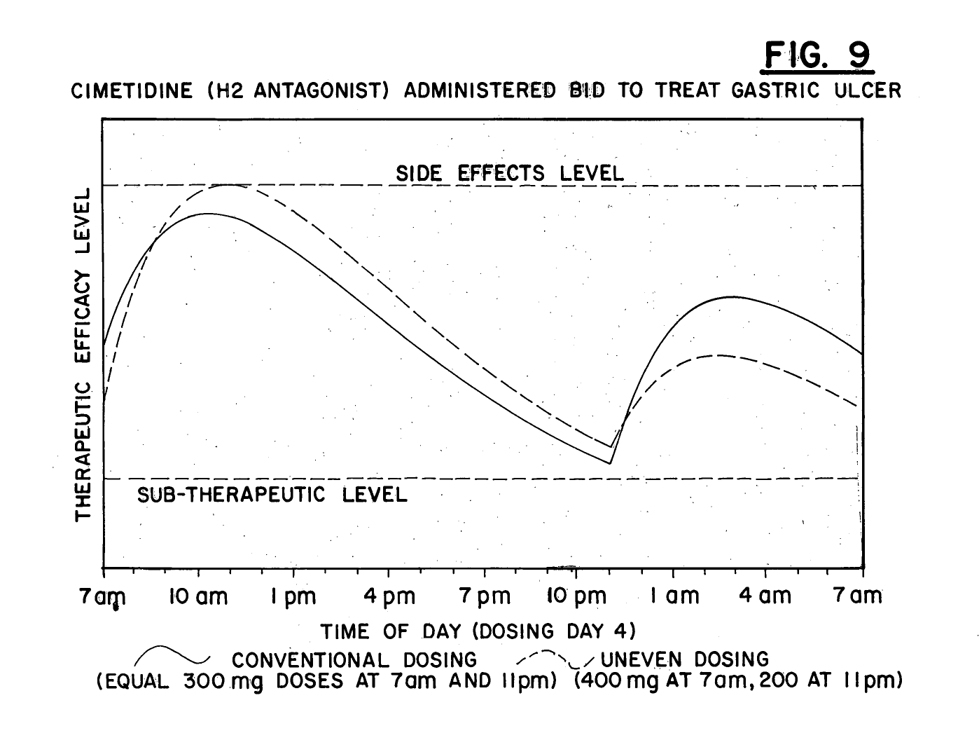

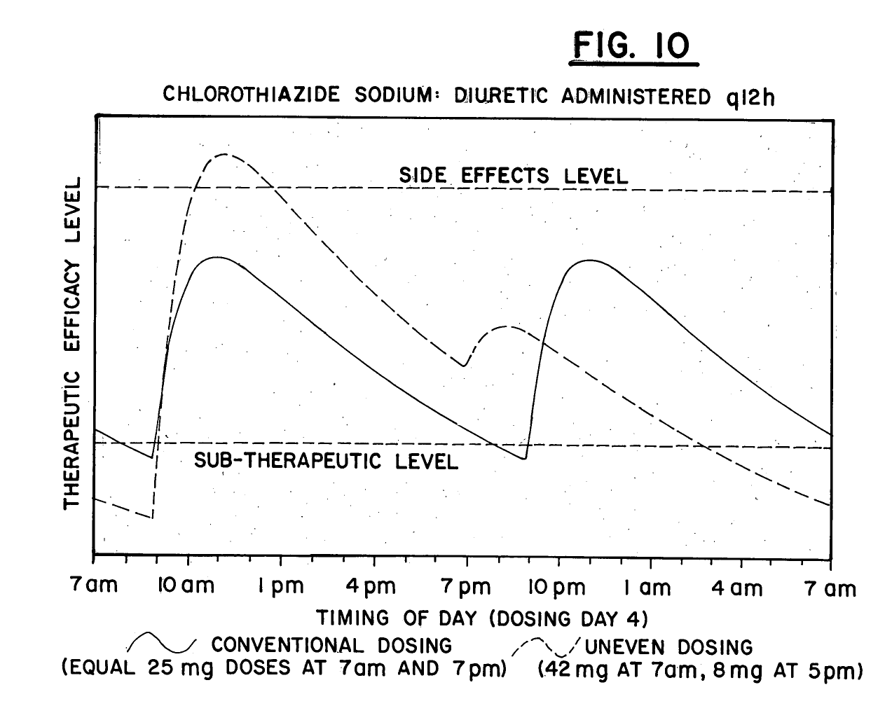

Application of U.S. patent application Ser. No. 09/053,487, filed Apr. 2, 1998, now U.S. Pat. No. 5,945,123, the entire contents of which are hereby incorporated by reference in their entirety.

### FIELD OF THE INVENTION

This invention relates to novel dosage forms, drug delivery regimens, methods and compositions which optimize therapeutic effects of biologically useful substances. The dosage forms, regimens, methods and pharmaceutical compositions of the present invention are adaptable to most biologically useful substances, and will improve the effectiveness of said substances. Particularly suitable substances include, without limitation, anti-hypertensive agents, osteoporotic agents, GERD agents, anti-viral agents, anti-neoplastic agents, inhaled steroids, anti-asthmatics, hormone replacement agents, anti-infectives, anti-diabetics, lipid lowering agents, thrombolytic agents, anticoagulant agents, fibrinolytic agents, nutritional agents, vitamins, minerals, electrolytes, herbal agents and fatty acids.

The present invention is particularly useful for adaptation to the specific schedules, cycles and needs of individuals, thereby frequently improving compliance with their therapy, reducing amounts required daily to less than conventionally utilized, and minimizing undesired effects commonly experienced.

### DESCRIPTION OF THE PRIOR ART

The administration of a substance(s) to achieve a therapeutic objective generally requires the attainment and maintenance of a biologic response, which in turn requires an appropriate concentration of the active substance(s) at a site or sites of action. The appropriate dosage needed to achieve a therapeutic objective largely depends upon factors specific to the individual being treated, such as the individual's clinical state, the severity of the condition being treated, and the presence of other drugs and concurrent disease. Further, a proper dosage also depends upon factors specific to the individual substance(s) being administered. These drug specific factors are characterized through two concepts: pharmacodynamics and pharmacokinetics.

Pharmacodynamics refers to the biologic response observed relative to the concentration at the active site. Pharmacokinetics refers to the attainment and maintenance of the appropriate concentration. Generally, once an individual's condition has been assessed and a substance(s) is chosen for administration, a dosage amount will be selected by taking into consideration the known pharmacokinetic parameters of the substance(s) in view of the individual's specific needs.

A substance(s) may be administered to the individual in a number of dosage forms. For example, the dosage may be administered in multiple doses throughout a 24 hour period, e.g., twice a day or three times a day. Further, the dosage may be administered in immediate release, controlled release, sustained release, timed release, delayed release, extended release, long acting and other such forms. Regardless of which of the above forms is employed, presently used dosage forms generally fail to account for the effects of administration between time intervals of differing lengths, the time at which doses are administered, and the varying physiological needs of individuals throughout the course of a day.

For example, a common dosing regimen described in the medical literature is the 9-1-5-9 regimen in which equal doses of a drug are administered once every four hours during the 12 daylight hours of a 24 hour period (e.g., at 9:00 am, 1:00 pm, 5:00 pm and 9:00 pm), and no doses are administered during the following 12 nighttime hours. See The Merck Manual, Sixteenth Edition, 277:2623 (1992). Therefore, in the 9-1-5-9 regimen, an individual will receive the same amount of active therapeutic substance(s) at 9:00 pm as at each of the other administrations, despite the substantially longer time interval of 12 hours following the 9:00 pm administration relative to the 4 hour time intervals following the other administrations.

Another common dosing regimen is that in which an individual takes one dose upon awakening and a second dose upon retiring. In this common twice-a-day regimen, sixteen hours may elapse between the daytime dose (6:00 AM to 10:00 PM) and only eight hours (10:00 PM to 6:00 AM) until the next dose is taken upon arising the next morning. Therefore, the individual will have either too high a dose during the night, or too low a dose during the day because the doses are equal.

Currently employed dosage forms, such as the ones described above, are problematic for a number of reasons. First, the administration of equal doses for time intervals of differing lengths results in levels of active therapeutic substance(s) at the site or sites of action which may be alternatively too high or too low to maintain therapeutic effectiveness over a given period of time.

Secondly, the currently employed dosage forms involve the administration of even doses at uneven time intervals thereby failing to account for physiological anomalies which occur throughout the course of a given 24 hour period. For example, conventional dosage forms fail to recognize the difference in an individual's metabolic rate during that individual's sleeping and waking hours.

Thirdly, currently used dosage forms will generally result in the administration of higher amounts of drug to a patient over a given period of time, which will in turn result in increased incidents of side effects. Further, as the body adapts to the presence of the higher amounts of active therapeutic substance(s), said therapeutic substance(s) will likely be less efficacious.

Fourthly, currently used dosage forms fail to factor into consideration the effects of the varying solubilities of their components. For example, in currently employed drug dosage forms a therapeutic substance containing a water-soluble component and a non water-soluble component would have equal amounts of water-soluble component present in each dose. Therefore, a tablet to be administered just prior to bedtime, for example, would contain the same dose of water-soluble substance(s) as a tablet to be administered in the morning dose. Such a dosing form fails to account for the specific absorption of each component at various times and again may result in levels of active therapeutic substance(s) at the site or sites of action which are either too high or too low at various times throughout a given 24 hour period.

In addition to the importance of the dosage forms for maintaining therapeutically effective drug levels at the site or sites of action, the success of a dosing form in achieving its therapeutic objective is largely dependent upon an individual's compliance with his or her drug dosing regimen. A individual's failure to comply with a dosing regimen, e.g. failure to take one or more doses of a drug or taking too many doses, will have an adverse impact upon the success of the regimen. Individuals may fail to comply with their drug dosing regimen for a number of reasons. For example, drug dosing regimens, such as the 9-1-5-9 regimen described above involve a rigid dosing schedule that may be incompatible with an individual's personal schedule. Such a rigid dosing schedule when combined with normal human traits such as forgetfulness or denial of medical condition, as well as a busy life, represent substantial obstacles to compliance with a drug dosing regimen. Accordingly, such rigid dosing regimens often result in the failure by an individual to take one or more doses at the prescribed time. This has an adverse impact on the levels of the therapeutic substance(s) at the active site or sites and consequently on the overall efficacy of the therapeutic substance(s).

Methods for optimizing the therapeutic effects of therapeutic substances by improving patient compliance with dosage regimens have been described. York, U.S. Pat. No. 5,521,208, describes novel compositions containing non-racemic mixtures of enantiomers tailored specifically to allow less frequent dosing and thus a more convenient dosing regimen to improve patient compliance of metabolically impaired individuals, such as individuals suffering from diabetes mellitus.

Lieberman et al., U.S. Pat. No. 5,597,072, describe a totally interactive patient compliance method which encourages compliance by a patient with their drug therapy by requiring that the patient call a phone number to obtain a code which will allow the patient to remove their medication from a specially designed dispenser and by recording each such phone call to signal that the patient has complied with the regimen.

Batchelor, U.S. Pat. No. 4,889,238, discloses a medicament package designed to improve compliance with a complex therapeutic regimen by providing blister packs containing the various medications to be administered and arranged in the order of their intended use.

Methods for optimizing the therapeutic effects of drugs by monitoring patients have also been described. Kell, U.S. Pat. Nos. 5,652,146 and 5,547,878, discloses a method of monitoring compliance of a patient that has been placed on a medication maintenance program with prescribed medication by determining a normalized urine medication concentration and comparing same to an expected medication concentration for an average patient.

Baggett, U.S. Pat. No. 4,811,845, discloses a medication compliance procedure and packaging system designed to ensure that a patient receives accurate doses of the required medication at scheduled times. The system involves a package indicating the time when each medication should be taken.

However, the above methods for improving patient compliance and monitoring patient compliance, would not alone optimize the efficacy of therapeutic substances and thus would not compensate for the previously described deficiencies of current drug dosage forms. Moreover, in the vast majority of cases, the above described methods for improving patient compliance or monitoring patients would not be appropriate because they are too costly or time consuming and because they are applicable to only a limited number of specific therapeutic substances, therapies, conditions or situations.

Dividing the total daily dosage of a drug into uneven multiple dosages has been previously described in the medical literature. For example, it has been disclosed that Sinemet®, a medication for treating Parkinson's disease, may be administered three times a day with each of the first two doses containing 300 mg of the medication and the third dose containing 200 mg of the medication. See Physicians' Desk Reference (PDR), Fifty First Edition, 959-963 (1997). Also disclosed in the medical literature is that subsequent to initiating a patient on Dilantin®, a medication for treating epilepsy, “the dosage may be adjusted to suit individual requirements”. See Physicians' Desk Reference (PDR), Fifty First Edition, 1965-1970 (1997). The medical literature also discloses that when administering Depakote®, a medication effective in treating migraines, mania or epilepsy, after an initial dosage of 750 mg daily, the dosage should be increased rapidly until the desired clinical effect or plasma concentration is achieved. See Physicians' Desk Reference (PDR), Fifty First Edition, 418-422 (1997).

However, these uneven dosage forms, as described in the medical literature, involve starting doses and arbitrary dose amounts which are not directed to all uses of a standardized dosage form for the purpose of achieving predictable concentrations of active therapeutic substance(s) at a site or sites of action, or plasma concentrations that would be associated with optimal therapy. Further, the uneven dosage forms described in the medical literature are associated with endpoint determinations or adjustments made in response to the clinical effects of the therapy. Moreover, the uneven dosage forms previously described do not recognize that the therapeutic window itself may change throughout the course of a day. For example, a patient may have different therapeutic need during the day than at night.

Another disclosure in the medical literature involves the administration of Ismo®, a medication administered for the prevention of angina pectoris due to coronary artery disease. According to the literature, Ismo® should be administered in two doses a day, only seven hours apart. Physicians' Desk Reference (PDR), Fifty First Edition, 2844-2845 (1997). However, this dosing schedule has been developed to minimize the impact of refractory tolerance and involves the use of equal doses in each administration of the drug.

The need to pattern administration of certain drugs to gradually increase blood level in a short period of time, often called titrating, has been recognized, as exemplified above. When titrating a patient, either a larger dose may be given in periods of the day or night when adverse symptoms climax, or smaller amounts may be given to reduce side effects such as sleeplessness. It is well known, however, that such methods of administration are designed to individualize dosing to each patient and do not deal with subsequent need to establish and maintain steady state. Conventionally, subsequent dosing is done once a day, twice a day, three times a day, four times a day or continuously.

In those instances where the prior art discloses applications of dissimilar doses, it is cited only for use in initially titrating patients and only for a limited number of disease states. The purpose of the prior art methods involving titration are to build plasma levels as quickly as possible. Dissimilar doses are used only incidentally to reach a desirable drug response. (Note, the contrast between “uneven dosing” as used herein in this patent where an a priori blood level has been anticipated based on the exactness of the uneven dose regimen.)

Therefore, there is a need for methods of treatment used not only to establish therapeutic effects, but also to achieve and maintain therapeutic effectiveness in steady state. There is also a need for methods of treatment which have universal applicability (i.e, the ability to be used in conjunction with a vast multitude of therapeutics). Whereas the prior art exists to provide pharmacological convenience and has limited applicability to a relatively short administration period, a need exists for methods useful in continued and prolonged treatment.

Further, a need remains for an easy and economical approach to achieving and maintaining levels of a therapeutic substance(s) known to be associated with optimal therapy and which can be applied to a limitless range of existing and future therapeutic and other substances. More specifically, a need remains for dosage forms, regimens, methods and compositions which account for uneven time intervals between doses, as well as daily physiological anomalies, and which can be administered in a more convenient manner. Such dosage forms, would be highly desirable in that they would improve compliance with the dosing regimen, while at the same time optimizing the therapeutic effects of the active therapeutic substance(s) being administered. Another desirable aspect of such dosage forms are that they would reduce the overall amount of therapeutic substance or substances administered and therefore minimize incidents of side effects and further optimize therapeutic effects.

### SUMMARY OF THE INVENTION

In the case of multiple dosing, it is well known that patients do not space doses evenly. Twice a day dosing may be instituted by the patient at 7:00 am and 12:00 pm. The first dose is thus required to provide the desired therapeutic effect for sixteen hours and the second like dose for eight hours. The plasma concentration profile which will result from repeated dosing on a similar schedule is shown in FIG. I which assumes a drug half-life of 12 hours.

The present invention recognizes the inconsistency, inadequacy, and dangers of such conventional dosing and provides flexible means to better assure compliance, maintain more even plasma levels, and reduce incidents of side effects. Generally, average daily requirement of therapeutic substance(s), as the result of such improved regulation of dosage, is reduced as well.

The present inventive subject matter is based on the discovery that novel, uneven dosage forms provide a more even and predictable physiologic response, or more even and predictable plasma concentrations, over any given period of time than currently employed dosage forms, thus optimizing the effectiveness of said biologically useful substance(s). The novel dosage forms of the present invention account for the uneven time intervals between doses, as well as daily physiological anomalies, which currently employed dosage forms do not address. Specifically, it is possible using the dosing forms of the present invention to target particular drug levels at different times throughout the day in recognition that different levels of drug may be desirable at different times throughout a day.

The novel dosage forms of the present invention can be administered in a convenient manner to improve patient compliance. Further, the dosage forms can be applied to any biologically active useful substance or substances in any situation. The dosage forms also reduce the overall amount of biologically useful substance(s) required to be administered over a given period of time and therefore minimize incidents of side effects and further optimize therapeutic effects.

One embodiment of the present inventive subject matter is a drug delivery regimen comprising an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, an anti-asthmatic, a hormone replacement agent, an anti-infective, an anti-diabetic, a vitamin, a mineral, an electrolyte, a fatty acid, an herbal agent, and combinations thereof administered during at least one 24 hour period of time to provide effective therapeutic levels of the active therapeutic substance(s) at a site or sites of action in an animal over said period, wherein each individual dose is independently adjusted to be administered to optimize levels of the active therapeutic substance(s) at the site or sites of action for maximum efficacy, and wherein the dose amount at each administration is independently characterized by the formula TD(t)=CD(t)+RD(t), where t is the time at which the dose is to be administered, TD (therapeutic dose) is the therapeutically effective dose at time (t), CD (current dose) is the dose to be administered at time (t), and RD (residual dose) is the amount of active therapeutic substance(s) remaining from the previous dose administration.

An alternative embodiment of the present inventive subject matter is a drug delivery regimen comprising multiple doses of an active therapeutic substance(s) administered during at least one 24 hour period of time to provide effective therapeutic levels of the active therapeutic substance(s) at a site or sites of action in an animal over said period, wherein the active therapeutic substance(s) is selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, a vitamin, an anti-asthmatic, a hormone replacement agent, an anti-infective, an anti-diabetic, a mineral, an electrolyte, an herbal agent, a fatty acid and combinations thereof administered in uneven doses and over varying time intervals, and wherein the uneven doses and the varying time intervals are selected to optimize levels of the active therapeutic substance(s) at the site or sites of action for maximum efficacy.

A further embodiment of present inventive subject matter is a drug delivery regimen comprising multiple doses of an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, an anti-asthmatic, a hormone replacement agent, an anti-infective, an anti-diabetic, a vitamin, a mineral, an electrolyte, an herbal agent, a fatty acid and combinations thereof administered during at least one 24 hour period of time to provide effective therapeutic levels of the active therapeutic substance(s) at a site or sites of action in an animal over said period, and wherein each dose is independently calculated according to known pharmacokinetic parameters of the active therapeutic substance(s) with variations to account for physiological anomalies which occur during said period to optimize levels of the active therapeutic substance(s) at the site or sites of action for maximum efficacy.

A still further embodiment of the present inventive subject matter is a drug delivery regimen comprising amultiple active therapeutic substances selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, an anti-asthmatic, a hormone replacement agent, an anti-infective, an anti-diabetic, a vitamin, a mineral, an electrolyte, an herbal agent, a fatty acid and combinations thereof administered during at least one 24 hour period of time to provide effective therapeutic levels of the active therapeutic substances at a site or sites of action in an animal over said period, wherein each dose is independently tailored to optimize levels of the respective active therapeutic substances at the site or sites of action for maximum efficacy.

Another embodiment of the inventive subject matter is a method of enhancing the therapeutic effect of an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, a hormone agent, an anti-arthritic agent, an antibiotic agent, an analgesic agent, a central nervous system or psychotrophic agent, a vitamin, a mineral, an electrolyte, an herbal agent, a fatty acid and combinations thereof in an animal, which comprises:

(a) determining known pharmacokinetic parameters of the active therapeutic substance(s);

(b) determining a number of doses to be administered during a 24 hour period of time and determining a time at which each dose will be administered by considering both the animal's schedule and physiological anomalies during the 24 hour period; and

(c) independently calculating the amount of each dose in accordance with the equation

TD(t)=CD(t)+RD(t)

where t is the time at which the dose is to be administered, TD (therapeutic dose) is the therapeutically effective dose at time (t), CD (current dose) is the dose to be administered at time (t), RD (residual dose) is the amount of active therapeutic substance(s) remaining from the previous dose administration.

Yet another embodiment of the present inventive subject matter is a method for maximizing therapeutic effectiveness of an antihypertensive agent, which comprises: administering a first dose of the antihypertensive agent at a first preselected time during a twenty four hour period; administering a second dose of the antihypertensive agent at a second preselected time during the twenty four hour period; wherein said first dose is about 30% of the total amount of the antihypertensive agent to be administered during the twenty four hour period and the second dose is about 70% of the total amount of the antihypertensive agent to be administered during the twenty four hour period; and wherein said first preselected time is about 6-8 am and the second preselected time is about 6-8 pm.

A further embodiment of the present inventive subject matter is a method for maximizing therapeutic effectiveness of an osteoporotic agent, which comprises: administering a first dose of the osteoporotic agent at a first preselected time during a twenty four hour period of time to an animal; administering a second dose of the osteoporotic agent at a second preselected time during the twenty four hour period of time to the animal; wherein said first dose is about 25% to about 35% of the total amount of the osteoporotic agent to be administered during the twenty four hour period of time and the second dose is about 65% to about 75% of the total amount of the osteoporotic agent to be administered during the twenty four hour period of time; and wherein said first preselected time is the period between the animal's awakening until just after the animal's morning meal and the second preselected time is the period between the animal's evening meal and the animal's bedtime.

An even further embodiment of the present inventive subject matter is a method for maximizing therapeutic effectiveness of AZT, which comprises: administering a first dose of AZT at a first preselected time during a twenty four hour period of time to an animal; administering a second dose of AZT at a second preselected time during the twenty four hour period of time to the animal; administering a third dose of AZT at a third preselected time during the twenty four hour period of time to the animal; wherein said first dose and the third dose are each equal and the second dose is 125-200% higher; and wherein said first preselected time is from 6 am to 9 am, the second preselected time is from 3 pm to 6 pm and the third preselected time is from 9 pm to 12 pm.

Yet another embodiment of the present inventive subject matter is a pharmaceutical composition for optimizing therapeutic activity, which comprises: a first active therapeutic substance(s) selected from the group consisting of water-soluble vitamins, water-soluble minerals and water-soluble electrolytes; and a second active therapeutic substance(s) selected from the group consisting of non water-soluble vitamins, non water-soluble minerals and fatty acids; wherein the ratio of the first active therapeutic substance(s) to the second active therapeutic substance(s) is independently tailored to optimize levels of the respective active therapeutic substances at a site or sites of action in an animal for maximum efficacy, and wherein said weight ratio is determined according to the time at which said composition is to be administered with a suitable pharmaceutical carrier.

Thus, the present inventive subject matter optimizes the therapeutic effectiveness of any active therapeutic substance(s). In particular, the present inventive subject matter optimizes the therapeutic effectiveness of active therapeutic substances selected from the group consisting of anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, a vitamin, a mineral, an electrolyte, an herbal agent, a fatty acid and combinations thereof.

### BRIEF DESCRIPTION OF THE DRAWINGS

FIG. I shows the expected results of the application of this invention to Methylphenidate administered to treat Attention Deficit Disorder (ADD). See Example I.

FIG. II shows the expected results of the application of this invention to Methylphenidate administered to treat Narcolepsy. See Example II.

FIG. III shows the expected results of the application of this invention to Vitamin B₁₂ administered for general health maintenance. See Example III.

FIG. IV shows the expected results of the application of this invention to Benzodiazipine administered to treat anxiety. See Example IV.

FIG. V shows the expected results of the application of this invention to terazosin hydrochloride, available from Abbott Laboratories under the tradename Hytrin, administered to prevent hypertension and heart attack. See Example V.

FIG. VI shows the expected results of another application of this invention to terazosin hydrochloride, available from Abbott Laboratories under the tradename Hytrin, administered to prevent hypertension and heart attack. See Example VI.

FIG. VII shows the expected results of the application of this invention to verapamil hydrochloride administered to prevent hypertension and heart attack. See Example VII.

FIG. VIII shows the expected results of the application of this invention to Cimetidine administered for the prevention of Gastroesophageal Reflux Disease (GERD). See Example VIII.

FIG. IX shows the expected results of the application of this invention to Cimetidine administered for the treatment of gastric ulcers. See Example IX.

FIG. X shows the expected results of another application of this invention to the diuretic Chlorothiazide Sodium administered to prevent hypertension. See Example X.

### DETAILED DESCRIPTION OF THE INVENTION

Definitions

As used herein, “Animal” refers to a human, mammal or any other animal.

“Drug delivery regimen” refers to the overall way in which a biologically useful substance(s) or active therapeutic substance(s) is administered to an animal.

“Substance”, “biologically useful substance” and “active therapeutic substance” refer to any substance or substances comprising a drug, active therapeutic substance, metabolite, medicament, vitamin or mineral, any substance used for treatment, prevention, diagnosis, cure or mitigation of disease or illness, any substance which affects anatomical structure or physiological function, or any substance which alters the impact of external influences on an animal, or metabolite thereof, and as used herein, encompasses the terms “active substance”, “therapeutic substance”, “agent”, “active agent”, “active therapeutic agent”, “drug”, “medication”, “medicine”, “medicant”, and other such similar terms.

“Site or sites of action” refers to the location at which an active therapeutic substance must be present to have its intended effect, and is synonymous with the term “active site or sites”.

“Effective therapeutic levels” refers to a range of levels of active therapeutic substance at a site or sites of action at which said active therapeutic substance will achieve its intended effect.

“Optimize” refers to the attainment of a level that falls within the range of levels at which therapeutically effective levels are achieved with little or no side effects.

“Maximum efficacy” refers to the highest amount of therapeutic effectiveness attainable with a specific active therapeutic substance.

“Therapeutic dose” is the range of levels of therapeutic substance required at the site or sites of action to achieve the intended effect of said therapeutic substance, and is synonymous with the term “therapeutically effective dose”.

“Therapeutic window” refers to the range of plasma concentrations, or the range of levels of therapeutically active substance the site or sites of action, with a high probability of therapeutic success.

“Plasma concentration” refers to the concentration of an active therapeutic substance in blood plasma.

“Drug absorption” refers to the process of movement from the site or sites of administration toward the systemic circulation.

“Bioavailability” refers to the rate at which an active moiety (drug or metabolite) enters the general circulation, thereby gaining access to a site or sites of action. “Chemical (pharmaceutical) equivalence” refers to drug substances that contain the same compound in the same amount and that meet current official standards; however, inactive ingredients in the drug substances may differ.

“Bioequivalence” refers to chemical equivalents that, when administered to the same individual in the same dosage regimen, result in equivalent concentrations of drug in blood and tissues.

“Therapeutic equivalence” refers to two drug substances that, when administered to the same individual in the same dosage regimen, provide essentially the same therapeutic effect or toxicity; they may or may not be bioequivalent.

“Drug Elimination” refers to the sum of the processes of drug loss from the body.

“Metabolism” refers to the process of chemical alteration of drugs in the body.

“Pharmacodynamics” refers to the factors which determine the biologic response observed relative to the concentration of drug at a site or sites of action.

“Pharmacokinetics” refers to the factors which determine the attainment and maintenance of the appropriate concentration of drug at a site or sites of action.

“Half-life” refers to the time required for the plasma drug concentration or the amount in the body to decrease by 50%.

The present inventive subject matter utilizes blood level data and clinical observations to show that conventional methods of dosing result in plasma levels which are often inconsistent with therapeutic need. Further, the present invention provides a simple mathematical means to usefully predict results of dosing. This has led to the non-obvious discovery that, by altering dosage forms and dosing regimens, less therapeutic substance(s) can be dosed to provide uniform therapeutic effectiveness or non-uniform effectiveness patterned to physiologic need and reduced incidence of side effects.

Further, the present inventive subject matter recognizes that the administration of equal doses of an active therapeutic substance(s) for time intervals of differing length results in levels of active therapeutic substance(s) at the site or sites of action which are alternatively too high or too low to consistently maintain therapeutic effectiveness over a given period of time. Moreover, a regimen involving the administration of such doses is particularly susceptible to physiological anomalies, such as changes in metabolism, throughout the course of any 24 hour period of time. It has been found that by tailoring each individual dose of an active therapeutic substance(s) to the time interval for which said substance(s) is to be administered and the time of day at which each dose is to be administered, more even therapeutically effective levels of said substance(s) at the site or sites of action, or more even plasma concentrations associated with optimal therapy, are achieved over time. Consequently, by tailoring each individual dose independently of the other doses, improved efficacy, and reduced side effects, are attained relative to currently employed even dosage forms.

It has been unexpectedly discovered that uneven dosing of biologically useful substances will maintain more uniform blood levels and systemic effects when the dosing is patterned to the uneven intervals in which these substances are administered, and the differing time related biochemical needs that may be time related, oftentimes with lower daily doses required because of the sparing effect which can result from such uneven dosing.

Dosing intervals are conventionally QD (once a day), BID (twice a day), TID (three times a day), QID (four times a day) or more frequent. Time of administration is based on half-life, formulation of the dosage form being utilized, systemic reactivity, convenience, whether self administered or regimented, and whether the substance(s) is therapeutic, nutritional, steroidal, or anti-infective.

Unless a substance is controlled released, or has a long half-life permitting QD administration, the time interval between ingestion of doses is ordinarily uneven. For example, if a substance is ingested upon arising and when retiring, the intervals are probably 16 and 8 hours. If taken upon arising, mid-day, and when retiring, intervals may be 5, 11 and 8 hours. If taken evenly spaced during awake hours, intervals might be 5.33, 5.33, 5.33 and 8 hours. In such cases, rational dosing should be uneven to be consistent with uneven time intervals.

Nutritionals and certain drugs, and steroids, antibiotics and like substances may best be taken on a full stomach. Such daytime intervals may be uneven and time between last daytime dose and next morning dose different.

When drugs, nutritionals, antibiotics and other therapeutic substances are administered parenterally (via drip system), therapeutic need and nursing convenience may give rise to intervals of administration that are unevenly spaced. The dose beginning a long period before the next dose is given should be larger than that of a following short period if uniform effects are desired. If it is desirable to establish higher blood levels during a daytime or night-time period, again the dosing should be uneven.

In administrating liquids, parenteral, salves, orifice preparations such as ointment, suspensions and liquids, measuring devices are used which facilitate uneven dosing. In the case of tablets, molded substances, or capsules, the dosage form should be adaptable to uneven dosing. Units having different dose levels can be prepackaged, for example in blister packs, and labeled for time of ingestion. Intervals can be BID, TID, QID or more frequent. In the case of capsules, one or more delayed action pellets can be included with long acting beads. Undoubtedly there are other alternative ways to formulate. As an example, long acting microparticles and suitable amounts of one or more amounts of particles with more delayed action microparticles may be mixed and encapsulated. Matrix substrates can be used to form 2, 3 or 4 multilayered tablets or press coated tablets. Press coated tablets can have delayed action cores. Differently formulated multilayered and press coated tablets, which may include coated and uncoated tablets packaged to specify time of use, can be used. Long acting and delayed action microparticles can likewise be suspended in parenteral fluids to provide uneven dosing. The same principle can be applied to ointments and salves which can be blister packed to differentiate doses. The above dosage forms are examples of existing dosage forms that can be adapted to provide uneven dosing and benefit derived therefrom.

### DRUG DELIVERY REGIMENS OF THE INVENTION

According to a first aspect of the invention, a drug delivery regimen comprises an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a nutritional agent, a vitamin, a mineral, an electrolyte, an herbal agent, a fatty acid and combinations thereof administered during a 24 hour period of time to provide effective therapeutic levels of the active therapeutic substance(s) at a site or sites of action in an animal over said period, wherein each individual dose is independently adjusted to be administered to optimize levels of the active therapeutic substance(s) at the site or sites of action for maximum efficacy, and wherein the dose amount at each administration will be independently determined by the following formula:

TD(t)=CD(t)+RD(t)

where

t is the time at which the dose is to be administered, TD (therapeutic dose) is the therapeutically effective dose at time (t),

CD (current dose) is the dose to be administered at time (t), and

RD (residual dose) is the amount of active therapeutic substance(s) remaining from the previous dose administration.

The present invention contemplates the use of known pharmacodynamic and pharmacokinetic parameters for active therapeutic substances. The present invention recognizes that the pharmacokinetic behavior of most drugs may be summarized by parameters that relate variables to each other. These parameters are constants, although their values may differ from patient to patient and in the same patient under different conditions. The basic pharmacokinetic parameters and their defining relationships are shown in Table I below:

TABLE I

| | |
|---|---|
| Relationship | Parameter |

Absorption

| | | | | | |
|---|---|---|---|---|---|
| 1. | Rate of | = | Absorption | × | Amount remaining |
| | absorption | | rate constant | | to be absorbed |
| 2. | Amount | = | Bioavailability | × | Dose |
| | Absorbed | | | | |

Distribution

| | | | | | |
|---|---|---|---|---|---|
| 3. | Amount in | = | Volume of | × | Plasma drug |
| | Body | | Distribution | | concentration |
| 4. | Unbound drug | = | Fraction | × | Plasma drug |
| | in plasma | | Unbound | | Concentration |

Elimination

| | | | | | |
|---|---|---|---|---|---|
| 5. | Rate of | = | Clearance | × | Plasma drug |
| | elimination | | | | concentration |
| 6. | Rate of renal | = | Renal | × | Plasma drug |
| | excretion | | clearance | | concentration |
| 7. | Rate of | = | Metabolic | × | Plasma drug |
| | metabolism | | clearance | | concentration |
| 8. | Rate of renal | = | Fraction | × | Rate of |
| | excretion | | excreted | | elimination |
| | | | unchanged | | |
| 9. | Rate of | = | Elimination | × | Amount in |
| | elimination | | Rate Constant | | body |

Determination of the proper dosage for a particular situation is performed using well know procedures and techniques available to the ordinary skilled artisan. The present invention enables a person skilled in the art to determine the appropriate dosage amounts to satisfy a therapeutic need by incorporating either known pharmacological parameters or readily ascertainable pharmacological parameters for a specific active therapeutic substance(s).

Moreover, the present invention recognizes that successful drug therapy requires planning drug administration according to the needs of the individual. One traditional approach for achieving successful individualized drug administration involves empirically adjusting the drug dosage until the therapeutic objective is met. However, this approach is frequently inadequate because of delays or undue toxicity. See The Merck Manual, Sixteenth Edition, 277:2610 (1992). An alternative approach for achieving individualized administration involves initiating drug administration according to the expected absorption and disposition (distribution and elimination) of the drug in an individual. The expected absorption and disposition of the drug in an individual is determined by using the known pharmacokinetic parameters as a function of the age and weight of the individual. Both of the above methods or any other such methods, without limitation, may be employed in conjunction with the present invention.

The present invention could result in the lowering of overall dosage required for maintaining even therapeutically effective levels of an active therapeutic substance(s) at a site or sites of action over a given time period. This effect is termed the “sparing dosage phenomena”. The sparing dosage phenomena is particularly dramatic in the case of active therapeutic substances with a long half-life. One particularly beneficial aspect of the sparing dosage phenomena created by the present invention is that incidents of side effects are minimized and less drug is required to consistently achieve therapeutic levels.

In a preferred embodiment of the invention, the active therapeutic substance(s) is administered to minimize incidents of side effects.

Another beneficial aspect of the present invention is that a drug dosing regimen may be established which is most convenient for the patient. By individually tailoring each dose to the time interval for which it is administered and/or the time of day at which it is administered, less frequent dosing and greater convenience of dosing may be attained. A more convenient dosing schedule will improve patient compliance with the therapy.

It is also possible in the present drug dosage regimens to combine various forms of release, which include, without limitation, extended release, controlled release, timed release, sustained release, delayed release, long acting, and pulsatile delivery, with immediate release to deliver various active therapeutic substances over various rates of release. The ability to obtain extended release, controlled release, timed release, sustained release, delayed release, long acting, pulsatile delivery and immediate release characteristics is performed using well known procedures and techniques available to the ordinary skilled artisan. Each of these specific techniques or procedures does not constitute an inventive aspect of this invention.

The present invention contemplates an active therapeutic substance(s) selected from drug classes, including without limitation, an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent and combinations thereof.

The active therapeutic substance(s) may further be selected from drug subclasses, including without limitation a calcium channel blocker, an ACE inhibitor, an angiotensin II receptor antagonist, a beta-adrenoceptor antagonist, an alpha 1-adrenoceptor antagonists, an alpha 2-adrenoceptor antagonist, a diuretic, an oral GI prokinetic agent, an agent active against H. Pylori, a proton pump inhibitor, a H₂ histamine receptor antagonist, an antacid, a cytoxic agent, an anti-metabolite, a platinum-containing compound, an antibiotic derivative, a fluoropyrimidine, a nitrosourea, a vinca alkaloid, a nitrogen mustard derivative, an adjuvant biological response modifier, a nucleoside analog, a protease inhibitor, a nicotinic acid, an HMG CoA reductase inhibitor, a bile sequestration agent, a fibric acid derivative, a heparin-like agent, a clot buster agent, an aspirin-like agent, a platelet glycoprotein IIb, IIIa receptor antagonist, a guanfacine, a carbonic anhydrase inhibitor, a loop diuretic, a thiazide, a potassium sparing diuretic, a thromboxane inhibitor and combinations thereof.

The active therapeutic substance(s) may further be selected from specific generic drugs, including without limitation nifedipine, verapamil, nicardipine, diltiazem, isradipine, amlodipine, felodipine, nifedipine, bepridil, alendronate, etidronate, pamidronate, clodronate, tiludronate, residronate, ibandronate, beclomethasone dipropionate, budesonide, flunisolide, fluticasone propionate, mometasone furoate, triamcinolone acetonide, quinapril, ramipril, captopril, benazepril, fosinopril, lisinopril, moexipril, enalapril, losartan, sotalol, timolol, esmolol, carteolol, propanolol, betaxolol, penbutolol, metoprolol, labetalol, acebutolol, atenolol, bisoprolol, doxazosin, phenoxybenzamine, guanethidine, guanadrel, terazosin, prazosin, methyldopa, clonidine, cisapride monohydrate, metoclopramide, clarithromycin, tetracycline, amoxicillin, bismuth, metronidazole, omeprazole, lansoprazole, cimetadine, famotidine, nizatidine, ranitidine, roxatidine, zidovudine, azidothymidine, didanosine, zalcitabine, stavudine, lamivudine, saquinavir mesylate, ritonavir, indinavir, placlitaxel, cyclophosphamide, teniposide, methotrexate, cisplatin (cis-diaminedichlororoplatinum), carboplatin, oxaliplatin, adriamycin, bleomycin, dactinomycin, daunorubicin, doxorubicin, indarubicin, mytomycin, 5-FU (5-fluorouracil), FudR (5-fluoro-2′-deoxyuridine), Ara-C (arabinosylcytosine), BCNU (carmustine), streptozocin, vinblastine, vincristine, thiotepa, alpha-interferon, TNF (tumor necrosis factor), EPO (erythropoietin), rhG-CSF (recombinant human granulocyte colony-stimulating factor), IL-1 (interleukin-1), IL-2 (interleukin-2), monoclonal antibodies to tumor and immunologic targets, atorvastatin, cerivastatin, fluvastatin, lovastatin, pravastatin, simvastatin, colestipol, cholestyramine, clofibrate, gemfibrozil, enoxaparin, dalteparin, refludan, streptokinases, alteplase (TPA), tirofiban, eptifibatide, abciximab, estrogen and combinations thereof.

The active therapeutic substance(s) may be administered in one or more dosage form(s) consisting of the therapeutic substance(s) or multiple therapeutic substances and other ingredients formulated into a useable substance(s). Any pharmaceutically acceptable dosage form, and combinations thereof, is contemplated by the invention. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods, by a vaporizer and combinations thereof. The preparation of any of the above dosage forms is well known to those skilled in the art; all of which are incorporated herein by reference.

The present invention contemplates substances formulated for administration by any route, including without limitation, oral, buccal, sublingual, by implant, rectal, parenteral, topical, subcutaneous, inhalational, injectable, vaginal, dermal, transdermal, transmucosal, eyedrops and through any body orifice, including eyes and ears. The physicochemical properties of therapeutic substances, their formulations, and the routes of administration are important in absorption. Absorption refers to the process of drug movement from the site or sites of administration toward the systemic circulation. Most orally administered therapeutic substances are in the form of tablets or capsules primarily for convenience, economy, stability, and patient acceptance. They must disintegrate and dissolve before absorption can occur. Using the present invention with any of the above routes of administration or dosage forms is performed using well known procedures and techniques available to the ordinary skilled artisan.

The present invention contemplates the use of pharmaceutically acceptable carriers which may be prepared from a wide range of materials. Without being limited thereto, such materials include diluents, binders and adhesives, lubricants, plasticizers, disintegrants, colorants, bulking substances, flavorings, sweeteners and miscellaneous materials such as buffers and absorbents in order to prepare a particular medicated composition.

Binders may be selected from a wide range of materials such as hydroxypropylmethylcellulose, ethylcellulose, microcrystalline cellulose, or other suitable cellulose derivatives, povidone, acrylic and methacrylic acid co-polymers, pharmaceutical glaze, gums, milk derivatives, such as whey, starches and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, crotonic acid, propylene glycol, butyl phthalate, dibutyl sebacate, castor oil and mixtures thereof, without limitation. As is evident, the plasticizers may be hydrophobic as well as hydrophilic in nature. Water-insoluble hydrophobic substances, such as diethyl phthalate, diethyl sebacate and castor oil are used to delay the release of water-soluble drugs, such as potassium chloride. In contrast, hydrophilic plasticizers are used when water-insoluble drugs are employed which aid in dissolving the encapsulating film, making channels in the surface, which aid in drug release.

Preferably, the active therapeutic substance(s) is administered in one or more dosage form(s) independently selected from the group consisting of liquid, solution, suspension, emulsion, tablet, bi-layer tablet, capsule, soft gelatin capsule, caplet, lozenge, chewable tablet, effervescent tablet or powder, quick dissolving tablet, bead, powder, granules, dispersible granules, cachets, douche, suppository, cream, topical, inhalant, patch, particle inhalant, implant, ingestible, injectable, or infusion.

The dosage forms can be in the form of a bi-layer tablet composed of at least one immediate-release layer. Also, the multi-layer tablet can be coated for ease of administration or can be enteric coated to reduce any gastric irritation and the unpleasant “burping” produced by certain therapeutic substance(s)s, such as vitamins and minerals. Also, multi-particulate design of extended-release and immediate-release components can be enteric coated and compressed into a tablet or filled into hard or soft gelatin capsules. Further, the substance(s) may be coated for an unlimited variety of effects, such as for delayed release, extended release, timed release, sustained release, and combinations thereof, without limitation.

The dosage forms of the present invention involve the administration of an active therapeutic substance(s) or multiple active therapeutic substances in a single dose during a 24 hour period of time or multiple doses during a 24 hour period of time. The doses may be uneven in that each dose is different from at least one other dose. The present invention contemplates variations between doses to include different quantities of the total dose, different quantities or proportions of an individual therapeutic substance(s) or multiple therapeutic substances within a dose, or different quantities or proportions of a related group of therapeutic substances, such as water-soluble therapeutic substances, within a dose. The time intervals between the administration of each dosage may also be uneven in that the time interval between each dose is different from at least one other such time interval.

The active therapeutic substance(s) may be administered in uneven doses or the active therapeutic substance(s) may be administered at uneven time intervals over the course of a 24 hour period of time. An “uneven dose” contemplates any aspects of the doses which causes at least one dose to vary from one to another. Thus, uneven doses may vary as to the quantity of a specific therapeutic substance(s) or substances, as to the ratio of various therapeutic substances, or as to any other element, such as, the manner of release, e.g. controlled release versus immediate release. Thus uneven doses of two or more substances may encompass one component being used in equivalent amounts whereas another substance may be used in uneven amounts when used in combination. For example, a patient may be administered an AM dose and a PM dose, wherein the AM dose is larger or smaller than the PM dose. A patient may be administered, an AM dose and a PM dose, wherein the AM dose is for immediate release and the PM dose is administered for controlled release. Another example involves the administration of an AM dose and a PM dose, wherein the AM dose has a higher or lower amount of a water-soluble active therapeutic substance(s) present than that present in the PM dose. An AM dose and a PM dose may be administered, wherein the AM dosage has a higher or lower amount of a non water-soluble drug present than that present in the PM dosage. Further, two PM doses may be administered, wherein the first PM dose is administered immediately after dinner and the second PM dose is administered immediately prior to bedtime.

The dosage may also be adjusted for subsequent 24 hour periods of time. Further, the active therapeutic substance(s) may be substituted for another active therapeutic substance(s). Adjustments to the dosage and substitutions of therapeutic substances may be done in response to clinical effects or observations, patient complaints, monitoring studies or test results, or for any other reason.

The active therapeutic substance(s) of the present inventive subject matter can vary widely depending upon is the desired objective. The active therapeutic substance(s) may be described as a single entity or a combination of entities. Examples of useful active therapeutic substances include, drugs from all major categories, including for example, without limitation, analgesics, anti-inflammatories, antitussives, expectorants, decongestants, narcotics, antibiotics, bronchodilators, cardiovascular preparations, central nervous system drugs, oncological preparations, antivirals, hormonal preparations, nutritionals, metal salts, vitamins, minerals, electrolytes, herbal agents and fatty acids.

Non-limiting exemplary analgesics include acetaminophen, ibuprofen, flurbiprofen, ketoprofen, voltaren (U.S. Pat. No. 3,652,762), phenacetin and salicylamide. Non-limiting exemplary anti-inflammatories include naproxen and indomethacin. Non-limiting exemplary antihistamines include chlorpheniramine maleate, phenindamine tartrate, pyrilamine maleate, doxylamine succinate, phenyltoloxamine citrate, diphenhydramine hydrochloride, promethazine, brompheniramine maleate, dexbrompheniramine maleate, clemastine fumerate and triprolidine. Non-limiting exemplary antitussives include dextromethorphan hydrobromide and guiaifenesin. Non-limiting exemplary expectorants include guaifenesin. Non-limiting exemplary decongestants include phenylephrine hydrochloride, phenylpropanolamine hydrochloride, pseudoephedrine hydrochloride, ephedrine. Non-limiting exemplary narcotics include morphine, codeine and their derivatives, such as oxycodone, hydrocodone and hydromorphone. Non-limiting exemplary antibiotics include macrolides, penicillins and cephalosporins and their derivatives. Non-limiting exemplary bronchodilators include theophylline, albuterol and terbutaline. Non-limiting exemplary cardiovascular preparations include diltiazem, cardura, propanolol, nifedepine and clonidine. Non-limiting exemplary central nervous system drugs include thioridazine, diazepam, meclizine, ergoloid mesylates, chlorpromazine, carbidopa and levodopa. Non-limiting exemplary metal salts include potassium chloride and lithium carbonate. Non-limiting exemplary hormone preparations include estrogen derivatives, progesterone derivatives and testosterone derivatives. Non-limiting examples of laxatives include aloin, cellulose derivatives, polycarbofil and phenolpthalein. Non-limiting examples of muscles relaxants include metaxalone and methacarbamol.

The active therapeutic substance(s) may be water-soluble or non water-soluble. Non-limiting exemplary water-soluble vitamins include vitamin B₁, vitamin B₂, vitamin B₃, biotin, pantothenic acid, vitamin B₆, folate, vitamin B₁₂, vitamin C, derivatives and combinations thereof. Non-limiting exemplary minerals include sodium, potassium, calcium, phosphorus, magnesium, sulfur, iron, zinc, iodide, copper, molybdenum, chromium, fluoride, derivatives thereof and combinations thereof. Non-limiting exemplary electrolytes include sodium, potassium, magnesium, calcium, derivatives and combinations thereof. Non-limiting exemplary non water-soluble vitamins include vitamin A, vitamin D, vitamin E and vitamin K. Non-limiting exemplary non water-soluble minerals include chromium, ferric iron, molybdenum, boron, selenium, manganese, bioflavonoid, derivatives thereof and combinations thereof. Non-limiting exemplary fatty acids include linoleic acid, linolenic acid, arachidonic acid, eicopentaenoic acid, docosahexaenoic acid, derivatives thereof and combinations thereof.

Non-limiting exemplary herbals and herbal derivatives include agrimony, alfalfa, aloe vera, amaranth, angelica, anise, barberry, basil, bayberry, bee pollen, birch, bistort, blackberry, black cohosh, black walnut, blessed thistle, blue cohosh, blue vervain, boneset, borage, buchu, buckthorn, bugleweed, burdock, capsicum, cayenne, caraway, cascara sagrada, catnip, celery, centaury, chamomile, chaparral, chickweed, chicory, chinchona, cloves, coltsfoot, comfrey, cornsilk, couch grass, cramp bark, culver's root, cyani, cornflower, damiana, dandelion, devils claw, dong quai, echinacea, elecampane, ephedra, eucalyptus, evening primrose, eyebright, false unicorn, fennel, fenugreek, figwort, flaxseed, garlic, gentian, ginger, ginseng, golden seal, gotu kola, gum weed, hawthorn, hops, horehound, horseradish, horsetail, hoshouwu, hydrangea, hyssop, iceland moss, irish moss, jojoba, juniper, kelp, lady's slipper, lemon grass, licorice, lobelia, mandrake, marigold, marjoram, marshmallow, mistletoe, mullein, mustard, myrrh, nettle, oatstraw, oregon grape, papaya, parsley, passion flower, peach, pennyroyal, peppermint, periwinkle, plantain, pleurisy root, pokeweed, prickly ash, psyllium, quassia, queen of the meadow, red clover, red raspberry, redmond clay, rhubarb, rose hips, rosemary, rue, safflower, saffron, sage, St. Johnswort, sarsaparilla, sassafras, saw palmetto, scullcap, senega, senna, shepherd's purse, slippery elm, spearmint, spikenard, squawvine, stillingia, strawberry, taheebo, thyme, uva ursi, valerian, violet, watercress, white oak bark, white pine bark, wild cherry, wild lettuce, wild yam, willow, wintergreen, witch hazel, wood betony, wormwood, yarrow, yellow dock, yerba santa, yucca and combinations thereof. Herbal derivatives, as used herein, refers to herbal extracts, and substances derived from plants and plant parts, such as leaves, flowers and roots, without limitation. Preferably, the herbal or herbal derivative is black cohosh, licorice, false unicorn, siberian ginseng, sarsaparilla, squaw vine, blessed thistle, peppermint, spearmint, red raspberry, St. Johnswort, ginger, kola, hops, valerian, derivatives thereof and combinations thereof.

Derivatives, as used herein, include, without limitation, salts, alkaline salts, esters and combinations thereof. The salts and alkaline salts herein refer to those regularly used organic or inorganic salts which are acceptable for pharmaceutical use.

Particularly preferred dosage forms involve the use of an active therapeutic substance(s) selected from the group consisting of Sinemet(r), levodopa, carbidopa, Eldepryl(r), selegiline, and combinations thereof; Ritalin(r), methylphenidate, and combinations thereof; nitroglycerin, disopyramide, nifedipine, and combinations thereof; antitussives, decongestants, and combinations thereof.

The present inventive subject matter may be used to treat, cure, prevent, control or alleviate a wide range of conditions and symptoms. For example, without limitation, in accordance with the present inventive subject matter, therapeutic agents may be administered to treat hypertension and other cardiovascular disorders, cancer, osteoporosis, gastroesophageal reflux disorder (GERD), vitamin and/or mineral deficiency, Parkinson's Disease, Attention Deficit Disorder. (ADD), cold/flu symptoms, bacterial and viral infections, pain, childhood bronchial asthma, peptic ulcer and post-operative recuperation.

The present inventive subject matter may also be used, without limitation, for improving overall health and in nutritional supplementation. The present inventive subject matter may be used with any vitamin and/or mineral supplements, for example, without limitation, vitamin and mineral supplements tailored to specific life stages and genders, such as vitamin and mineral supplements for pregnant, lactating, non-lactating or menopausal women.

It is also contemplated that the present inventive subject matter may optionally further incorporate additional drug delivery regimens, methods, therapies and treatments.

The drug delivery regimens contemplate that each individual dose may be predetermined and therefore independently adjusted without regard for endpoint determinations. In a particularly preferred embodiment of the invention, each individual dose is independently adjusted without regard for an endpoint determination.

Preferably, the drug delivery regimen comprises multiple doses of an active therapeutic substance(s) administered during at least one 24 hour period of time to provide effective therapeutic levels of the active therapeutic substance(s) at a site or sites of action in an animal over said period, wherein the active therapeutic substance(s) is administered in uneven doses and over varying time intervals, and wherein the uneven doses and the varying time intervals are selected to optimize levels of the active therapeutic substance(s) at the site or sites of action for maximum efficacy.

More preferably, the drug delivery regimen comprises multiple doses of an active therapeutic substance(s) administered. during at least one 24 hour period of time to provide effective therapeutic levels of the active therapeutic substance(s) at a site or sites of action in an animal over said period, and wherein each dose is independently calculated according to known pharmacokinetic parameters of the active therapeutic substance(s) with variations to account for physiological anomalies which occur during said period to optimize levels of the active therapeutic substance(s) at the site or sites of action for maximum efficacy.

Even more preferably, the drug delivery regimen comprises multiple doses of an active therapeutic substance(s) administered during at least one 24 hour period of time to provide effective therapeutic levels of the active therapeutic substance(s) at a site or sites of action in an animal over said period, wherein the time at which each dose is to be administered is tailored to a convenient schedule for the animal, and wherein the dose amount at each administration will be independently determined by the formula TD(t)=CD(t)+RD(t), where t, TD, CD and RD are as defined above.

Most preferably, the drug delivery regimen of the invention comprises multiple active therapeutic substances administered during at least one 24 hour period of time to provide effective therapeutic levels of the active therapeutic substances at a site or sites of action in an animal over said period, wherein each dose is independently tailored to optimize levels of the respective active therapeutic substances at the site or sites of action for maximum efficacy.

Another aspect of the present invention recognizes that certain types of therapeutic substances exhibit different pharmacodynamic and pharmacokinetic characteristics than others at various times during a 24 hour period of time. For example, it is known that water-soluble B vitamins are used in nervous tissue regeneration, which occurs mainly during sleep. A high morning dose of the water soluble B group of vitamins is excreted rapidly, before having any effect. The present invention accounts for these time sensitive characteristics by varying the proportion of the substances from dose to dose when appropriate. Therefore, in accordance with the present invention, one would divide the B vitamin dose so that a much smaller quantity of B vitamin is present in the A.M. as compared to a much larger quantity in the P.M. This represents a departure from currently employed dosage forms which contain substances in the same proportion from dose to dose.

In another. example, it is known that calcium is often used in bone marrow regeneration, which mainly occurs at night. A high morning dosage of calcium is excreted rapidly, before it can have any effect. The present invention accounts for these time sensitive characteristics by varying the proportion of the substances from dose to dose when appropriate. Therefore, in accordance with the present invention, one would divide the calcium dose so that a much smaller quantity of calcium is present in the A.M. as compared with a much larger quantity in the P.M.

Preferably, the drug delivery regimen comprises multiple active therapeutic substances administered over a 24 hour period of time to provide effective therapeutic levels of the respective active therapeutic substances over said period, wherein the ratio of active therapeutic substances to each other for each individual dose will be independently tailored to optimize levels of the active therapeutic substance(s) at the site or sites of action for maximum efficacy.

More preferably, the drug delivery regimen comprises multiple active therapeutic substances administered over a 24 hour period of time to provide effective therapeutic levels of the active therapeutic substance(s) at a site or sites of action in an animal over said period, wherein the ratio of the therapeutic substances to each other for each dose will not equal the ratio of the therapeutic substance(s) to each other for at least one of the other doses, and wherein the ratio of therapeutic substances to each other for each individual dose is independently tailored to optimize levels of the active therapeutic substance(s) at the site or sites of action for maximum efficacy.

Even more preferably, the drug delivery regimen comprises an active therapeutic substance(s) with a water-soluble phase and a non water-soluble phase administered during at least one 24 hour period of time to provide effective therapeutic levels of the active therapeutic substances at a site or sites of action in an animal over said period, wherein the ratio of water-soluble phase to non water-soluble phase for each dose is independently tailored to optimize levels of the active therapeutic substance(s) at the site or sites of action for maximum efficacy.

Most preferably, the drug delivery regimen comprises an active therapeutic substance(s) with a water-soluble phase and a non water-soluble phase administered over a 24 hour period of time to provide effective therapeutic levels of the active therapeutic substance(s) at a site or sites of action in an animal over said period, wherein for each individual dose the ratio of the water-soluble phase to the non water-soluble phase will not equal the ratio of the water-soluble phase to the non water-soluble phase for at least one of the other doses, and wherein the ratio of water-soluble phase to non water-soluble phase for each individual dose will be independently tailored to optimize levels for maximum efficacy.

Administration of the active therapeutic substance(s) includes, without limitation, administration of the active therapeutic substance(s) by the individual to whom said substance(s) is being administered (i.e. self-administration), administration by a medical professional to a patient, or administration by any party assisting another party with the taking of said substance(s) (i.e., a parent administering medication to his or her child or a family member administering medication to an elderly relative).

As described above, the present invention encompasses several different inventive means which are all achieved using the methodology set forth herein. For example, one inventive means assures compliance to dosing regimens by providing dosage forms so formulated that a majority of therapeutic substances, heretofore administered twice a day, three times a day, four or more times a day, can be ingested upon arising and when retiring; the most convenient and most easily remembered times in a twenty four hour day. Another inventive means involves formulating and administering the therapeutic substances to provide more uniform therapeutic effects when ingested in unequal amounts and uneven intervals, as well as the formulating and administering of therapeutic substances to provide non-uniform therapeutic effects when ingested in equal or unequal intervals to satisfy unequal needs.

The present invention also encompasses the formulating and administering of therapeutic substances, conventionally dosed once a day, in more than one dose to obtain more or less uniform blood concentrations patterned to uniform or non-uniform need and requires less total daily dosage which reduces possible incidence of side effects. Also encompassed by the present invention is the formulating and administering of therapeutic one or more therapeutic substance(s) twice, three times, or four times and day at other intervals than conventional intervals to obtain more optimal blood concentrations and consequent effectiveness. Accordingly, the administration of the therapeutic substance(s) will be more effective. Furthermore, the total amount of the therapeutic substance(s) administered each day may be reduced while still maintaining the same efficacy.

Although the dosage forms of the invention are preferably intended for humans, it will be understood that said dosage forms may also be utilized in veterinary therapies for other animals.

### METHODS OF THE INVENTION

Another aspect of the present invention is a method. of enhancing the therapeutic effect of an active therapeutic substance(s) selected from the group consisting of an anti-hypertensive agent, an osteoporotic agent, a GERD agent, an antibiotic agent, an anti-viral agent, an anti-neoplastic agent, an inhaled steroid, a lipid lowering agent, a thrombolytic agent, an anticoagulant agent, a fibrinolytic agent, a nutritional agent, a vitamin, a mineral, an electrolyte, an herbal agent, a fatty acid and combinations thereof in an animal, which comprises:

(a) determining known pharmacokinetic parameters of the active therapeutic substance(s);

(b) determining a number of doses to be administered during a 24 hour period of time and determining a time at which each dose will be administered by considering both the animal's schedule and physiological anomalies during the 24 hour period; and

(c) independently calculating the amount of each dose in accordance with the equation

TD(t)=CD(t)+RD(t)

where t, TD, CD and RD are as defined above.

Determination of the proper dosage for a particular situation is performed using well known procedures and techniques available to the ordinary skilled artisan. The present invention enables a person skilled in the art to determine the appropriate dosage amounts for a particular situation by incorporating either known biologic responses, pharmacological parameters or readily ascertainable pharmacological parameters for a specific active therapeutic substance(s).

Steps (a) and (b) can be performed by the ordinary skilled artisan using information readily available from medical literature or readily determinable using techniques available to the ordinary skilled artisan. The calculation in step (c) can be performed by the ordinary skilled artisan using the information gathered for steps (a) and (b) and using the known relationships between pharmacokinetic parameters. The precise calculations to be used will vary widely depending upon the situation and active therapeutic substance(s) or substances involved.

### COMPOSITIONS OF THE INVENTION

Another aspect of the invention includes compositions for optimizing therapeutic activity in an animal, which comprise: a substance consisting essentially of an active therapeutic substance(s) selected from the group consisting of an anti-hypertensive agent, an osteoporotic agent, a GERD agent, an antibiotic agent, an anti-viral agent, an anti-neoplastic agent, an inhaled steroid, a lipid lowering agent, a thrombolytic agent, an anticoagulant agent, a fibrinolytic agent, a nutritional agent, a vitamin, a mineral, an electrolyte, an herbal agent, a fatty acid and combinations thereof in dose amounts calculated according to the formula TD(t)=CD(t)+RD(t), where t, TD, CD and RD are as defined above in combination with a suitable pharmaceutical carrier.

Determination of the proper dosage for a specific composition is performed using well known procedures and techniques available to the ordinary skilled artisan. The present invention enables a person skilled in the art to determine the appropriate dosage amounts for a particular situation by incorporating either known pharmacological parameters or readily ascertainable pharmacological parameters for a specific active therapeutic substance(s).

Moreover, the present invention recognizes that successful drug therapy requires planning drug administration according to the needs of each individual. One traditional approach for achieving successful individualized drug administration involves empirically adjusting the drug dosage until the therapeutic objective is met. However, this approach is frequently inadequate because of delays or undue toxicity. See Merck Index, Chapter 277, p. 2610. An alternative approach for achieving individualized administration involves initiating drug administration according to the expected absorption and disposition (distribution and elimination) of the drug in an individual. The expected absorption and disposition of the drug in an individual is determined by using the known pharmacokinetic parameters as a function of the age and weight of the individual. Both of the above methods or any other such methods, without limitation, may be employed in conjunction with the present invention.

Another aspect of the present invention recognizes that certain types of therapeutic substances exhibit different pharmacodynamic and pharmacokinetic characteristics than others at various times during a 24 hour period of time. For example, it is known that water-soluble B vitamins are used in nervous tissue regeneration, which occurs mainly during sleep. A high morning dose of the water soluble B group of vitamins is excreted rapidly, before having any effect. The present invention accounts for these time sensitive characteristics by varying the proportion of the substances from dose to dose when appropriate. Therefore, in accordance with the present invention, one would divide the B vitamin dose so that a smaller quantity of B vitamin is present in the A.M. as compared to a larger quantity in the P.M. This represents a departure from currently employed dosage forms which contain substances in the same proportion from dose to dose.

In a particularly preferred embodiment of the invention, a pharmaceutical composition for optimizing therapeutic activity comprises a substance consisting essentially of multiple active therapeutic substances, wherein the substance has a water-soluble phase and a non water-soluble phase in combination with a suitable pharmaceutical carrier, and wherein the ratio of water-soluble phase to non water-soluble phase is independently tailored to optimize levels of the respective active therapeutic substances at a site or sites of action in an animal for maximum efficacy, and wherein said ratio is determined according to the time at which said composition is to be administered.

Tablets incorporating the above formulations are prepared using conventional methods and materials known in the pharmaceutical art. The resulting nutritional compositions were recovered and stored for future use.

The composition of the present invention may also include one or more biologically active substance(s). The biologically active substances incorporated into the present invention are nonteratogenic to protect the unborn fetus. For example, without limitation, the biologically active substance(s) may be a lactogen compound, a derivative of a lactogen compound or combinations thereof. Derivatives of lactogen compounds include, without limitation, salts of lactogen compounds, alkaline salts of lactogen compounds, esters of lactogen compounds and combinations thereof.

Various additives may be incorporated into the present composition. Optional additives of the present composition include, without limitation, starches, sugars, fats, antioxidants, amino acids, proteins, derivatives thereof or combinations thereof.

It is also possible in the nutritional composition of the present invention for the dosage form to combine various forms of release, which include, without limitation, immediate release, extended release, pulse release, variable release, controlled release, timed release, sustained release, delayed release, long acting, and combinations thereof. The ability to obtain immediate release, extended release, pulse release, variable release, controlled release, timed release, sustained release, delayed release, long acting characteristics and combinations thereof is performed using well known procedures and techniques available to the ordinary artisan. Each of these specific techniques or procedures for obtaining the release characteristics does not constitute an inventive aspect of this invention. As used herein, a “controlled release form” means any form having at least one component formulated for controlled release. As used herein, “immediate release form” means any form having all its components formulated for immediate release.

Any biologically-acceptable dosage form, and combinations thereof, are contemplated by the invention. Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods and combinations thereof. The preparation of the above dosage forms are well known to persons of ordinary skill in the art.

The following represent examples, without limitation, of acceptable methods of preparing some of the above-listed dosage forms. For example, animal feed may be by methods well known to persons of ordinary skill in the art. Animal feeds may be prepared by mixing the formulation with binding ingredients to form a plastic mass. The mass is then extruded under high pressure to form tubular (or “spaghetti-like”) structures that are cut to pellet size and dried.

Quick dissolve tablets may be prepared, for example, without limitation, by mixing the formulation with agents such as sugars and cellulose derivatives, which promote dissolution or disintegration of the resultant tablet after oral administration, usually within 30 seconds.

Cereal coatings may be prepared, for example, without limitation, by passing the cereal formulation, after it has been formed into pellets, flakes, or other geometric shapes, under a precision spray coating device to deposit a film of active ingredients, plus excipients onto the surface of the formed elements. The units thus treated are then dried to form a cereal coating.

For example, health bars may be prepared, without limitation, by mixing the formulation plus excipients (e.g., binders, fillers, flavors, colors, etc.) to a plastic mass consistency. The mass is then either extruded or molded to form “candy bar” shapes that are then dried or allowed to solidify to form the final product.

Soft gel or soft gelatin capsules may be prepared, for example, without limitation, by dispersing the formulation in an appropriate vehicle (vegetable oils are commonly used) to form a high viscosity mixture. This mixture is then encapsulated with a gelatin based film using technology and machinery known to those in the soft gel industry. The industrial units so formed are then dried to constant weight.

Chewable tablets, for example, without limitation, may be prepared by mixing the formulations with excipients designed to form a relatively soft, flavored, tablet dosage form that is intended to be chewed rather than swallowed. Conventional tablet machinery and procedures, that is both direct compression and granulation, i.e., or slugging, before compression, can be utilized. Those individuals involved in pharmaceutical solid dosage form production are well versed in the processes and the machinery used as the chewable dosage form is a very common dosage form in the pharmaceutical industry.

Film coated tablets, for example, without limitation, may be prepared by coating tablets using techniques such as rotating pan coating methods or air suspension methods to deposit a contiguous film layer on a tablet. This procedure is often done to improve the aesthetic appearance of tablets, but may also be done to improve the swallowing of tablets, or to mask an obnoxious odor or taste, or to improve to usual properties of an unsightly uncoated tablet.

Compressed tablets, for example, without limitation, may be prepared by mixing the formulation with excipients intended to add binding qualities to disintegration qualities. The mixture is either directly compressed or granulated then compressed using methods and machinery quite well known to those in the industry. The resultant compressed tablet dosage units are then packaged according to market need, i.e., unit dose, rolls, bulk bottles, blister packs, etc.

The present invention contemplates nutritional compositions formulated for administration by any route, including without limitation, oral, buccal, sublingual, by implant, rectal, parenteral, topical, subcutaneous, inhalational, injectable, vaginal, dermal, transdermal, transmucosal, eyedrops and through any body orifice, including eyes and ears. The physicochemical properties of nutritional compositions, their formulations, and the routes of administration are important in absorption. Absorption refers to the process of nutritional composition movement from the site or sites of administration toward the systemic circulation. Most orally administered nutritional compositions are in the form of tablets or capsules primarily for convenience, economy, stability, and patient acceptance. They must disintegrate and dissolve before absorption can occur. Using the present invention with any of the above routes of administration or dosage forms is performed using well known procedures and techniques available to the ordinary skilled artisan.

The present invention contemplates the use of pharmaceutically acceptable carriers which may be prepared from a wide range of materials. Without being limited thereto, such materials include diluents, binders and adhesives, lubricants, plasticizers, disintegrants, colorants, bulking substances, flavorings, sweeteners, edible oils, polymers and miscellaneous materials such as buffers and absorbents in order to prepare a particular medicated composition.

Binders may be selected from a wide range of materials such as hydroxypropylmethylcellulose, ethylcellulose, or other suitable cellulose derivatives, povidone, acrylic and methacrylic acid co-polymers, pharmaceutical glaze, gums, milk derivatives such as whey, starches, and derivatives, as well as other conventional binders well known to persons skilled in the art. Exemplary non-limiting solvents are water, ethanol, isopropyl alcohol, methylene chloride or mixtures and combinations thereof. Exemplary non-limiting bulking substances include sugar, lactose, gelatin, starch, and silicon dioxide.

The plasticizers used in the dissolution modifying system are preferably previously dissolved in an organic solvent and added in solution form. Preferred plasticizers may be selected from the group consisting of diethyl phthalate, diethyl sebacate, triethyl citrate, cronotic acid, propylene glycol, butyl phthalate, dibutyl sebacate, caster oil and mixtures thereof, without limitation. As is evident, the plasticizers may be hydrophobic as well as hydrophilic in nature. Water-insoluble hydrophobic substances, such as diethyl phthalate, diethyl sebacate and caster oil are used to delay the release of water-soluble vitamins, such as vitamin B₆ and vitamin C. In contrast, hydrophilic plasticizers are used when water-insoluble vitamins are employed which aid in dissolving the encapsulated film, making channels in the surface, which aid in nutritional composition release.

The compositions of the present invention contemplate formulations of various viscosities. The viscous stresses in liquids arise from intermolecular reaction. The concept of viscosity in relation to soft gelatin medicament formulations is important when it is considered that viscosity is used as an index of the suitability of a particular formulation for a particular purpose, i.e., the suitability of a biologically-active core for insertion into a soft gelatin shell.

The centipoise unit is frequently used to measure the dynamic viscosity of mobile liquids and is the unit basis contemplated by the present invention. The formal definition of viscosity is derived from a Newtonian theory, wherein under conditions of parallel flow, the shearing stress is proportional to the velocity gradient. If the force acting on each of the two planes of area A parallel each other, moving parallel to each other with a relative velocity V, and separated by a perpendicular distance X, be denoted by F, the shearing stress is F/A and the velocity gradient, which will be linear for a true liquid, is V/X. Thus, F/A=ηV/X, where the constant η is the viscosity coefficient or dynamic viscosity of the liquid. Van Nostrand's Scientific Encyclopedia, 2891 (6^{th} Ed. 1983).

The dosage forms of the present invention may be prepared as follows, for example, without limitation, by dispersing the formulation in an appropriate vehicle, such as vegetable oil or the like, to form a high viscosity mixture. Preferably the viscosity of the mixture would range from about 1,000 centipoise to about 1.5 million centipoise. This mixture is then encapsulated with a gelatin based film using technology and machinery known to the soft gel industry. The industrial units so formed are then dried to a constant weight and stored for future use.

The forgoing is considered as illustrative only of the principles of the invention. Further, since numerous modification and changes will readily occur to those skilled in the art, it is not desired to limit the invention to the exact construction and operation shown and described, and accordingly all suitable modifications and equivalents may be restored to, falling within the scope of the invention. The following examples are illustrative of preferred embodiments of the invention and are not to be construed as limiting the invention thereto.

An inexhaustible number of examples could be given to support all the ways uneven dosing can be utilized to improve the effectiveness of ingested substances. Nevertheless, the principles by which dosage and form are designed is always the same. To illustrate, the following figures show dose formation and effectiveness of QD, BID, TID and QID drugs, with expected half-lives, converted to uneven form for administration upon awakening and when retiring. For manufacturing a dispensing convenience, it is assumed tablets are used and two tablets are taken upon arising and one when retiring. Because of the sparing effect, a daily dose lower than the conventional daily dose is evaluated in some examples. A QD substance with too short a half-life to use BID was selected to demonstrate the solution to such a limitation. In such a case, QID drug is developed into a reduced dose relatively short duration long acting form and administered 2 to 1.

The present invention is further illustrated by the following specific examples which are not deemed to be limiting thereof. All amounts specified in the application are based on milligrams unless otherwise indicated. The term “I.U.” represents International Units. All percentages used throughout the specification and claims are based on the weight of the final product, unless otherwise indicated, and all formulations total 100% by weight.

### EXAMPLES

### Example I

The plasma profile for methylphenidate, available from CibaGeneva under the trade name Ritalin®, when administered in a conventional form, 10 mg at 7 am and 10 mg at 12 pm, for the treatment of Attention Deficit Disorder (ADD) was determined based on data available in the medical literature and is illustrated by the solid line in FIG. I. Note that when using the conventional administration, high dosages of the drug would be present in the body throughout the afternoon and early evening, causing over-stimulation of the patient and resultant side effects, such as twitching and convulsions.

A single dose of 20 mg Ritalin® was then administered to each of 6 normal adult males. After measuring plasma concentrations of the 6 normal adult males, an exemplary plasma profile for the drug, using uneven dosing, 14 mg at 7 am and 6 mg at 3 pm, was developed with a pharmacokinetic mathematical model, as illustrated by the dashed line in FIG. I. Note that the uneven dosing will result in more acceptable dosages of drug throughout the afternoon and early evening, thus avoiding side effects, while also providing higher dosages of drug in the morning, when the patient is most active and thus most susceptible to the symptoms of ADD.

### Example II

The plasma profile for methylphenidate, available from CibaGeneva under the trade name Ritalin®, when administered in a conventional form, with 20 mg at 7 am, 10 mg at 12 pm and 10 mg at 5 pm, for the treatment of narcolepsy was determined based on data available in the medical literature and is illustrated by the solid line in FIG. II. Note that when using the conventional administration, lower dosages of the drug are present in the patient during the morning hours when the patient has the greatest difficulty staying awake and increasingly higher dosages of the drug would be present in the body throughout the evening and bedtime hours, resulting in sleeplessness.

A single dose of 20 mg Ritalin® was then administered to each of 6 normal adult males. After measuring plasma concentrations of the 6 normal adult males, an exemplary plasma profile for the drug was developed with a pharmacokinetic mathematical model, using uneven dosing, 20 mg at 7 am and 10 mg at 3 pm, as illustrated by the dashed line in FIG. II. Note that the uneven dosing will result in higher levels of the drug in the patient during the morning hours, when the patient needs stimulation the most. Further, the uneven dosing will result in lower levels of drug in the evening and night, thus avoiding the sleeplessness that results from conventional dosing.

### Example III

The plasma profile for vitamin B₁₂, when administered in conventional form, 12 mcg at 7 am, is illustrated by the solid line in FIG. III. Note that when using the conventional administration, there is virtually no vitamin B₁₂ present in the patient during the evening and nighttime hours when nerve tissue repair, which is known to require vitamin B₁₂, predominantly occurs.

An exemplary plasma profile for vitamin B₁₂ is set forth using uneven dosing, 4 mcg at 7 am and 8 mcg at 11 pm, as illustrated by the dashed line in FIG. III. Note that the uneven dosing will result in the presence of high levels of vitamin B₁₂ in the patient during the nighttime hours, when the vitamin is most beneficial to the patient because it is available to assist in the repair of nerve tissue, that may be a result of stroke or other trauma.

### Example IV

The plasma profile for Benzodiazipine, available from Roche Products under the trade name Valium®, when administered in a conventional form, 10 mg at 7 am, 10 mg at 3 pm and 10 mg at 7 pm, for the treatment of anxiety, is illustrated by the solid line in FIG. IV. Note that when using the conventional administration, relatively low dosages of the drug are present in patients during the morning hours, when patients are most likely to experience the most severe symptoms of anxiety. Further, when using conventional administration, relatively high dosages of the drug are present during the nighttime hours when the symptoms of anxiety tend to be minimal.

An exemplary plasma profile for the same drug is set forth using uneven dosing, 20 mg at 7 am and 10 mg at 10 pm, as illustrated by the dashed line in FIG. IV. Note that the uneven dosing will result in relatively high levels of the drug in the patient during the morning hours, when symptoms tend to be most severe, and relatively low levels of the drug during the night when the symptoms tend to be least severe.

### Example V

The plasma profile for terazosin hydrochloride, available from Abbott Laboratories under the trade name Hytrin®, when administered in a conventional form, with even doses at 7 am and 7 pm, for the prevention of hypertension and heart attack, is illustrated by the solid line in FIG. V. Note that when using the conventional administration, unnecessarily high dosages of the drug are present in patients during the evening hours, when patients are least likely to experience a heart attack, and during the morning hours when most heart attacks occur, the dosage is lower than may be required.

An exemplary plasma profile for the same drug is set forth using uneven dosing, with two thirds of the total daily dosage administered at 7 am and one third of the total daily dosage administered at 10 pm, as illustrated by the dashed line in FIG. V. Note that the uneven dosing will result in relatively high levels of the drug in the patient during the morning hours, when the patient is most vulnerable to a heart attack, and relatively low levels of the drug during the evening when the patient is least vulnerable to a heart attack.

### Example VI

The plasma profile for terazosin hydrochloride, available from Abbott Laboratories under the trade name Hytrin®, when administered in a conventional form, with even doses at 7 am and 11 pm, for the prevention of hypertension and heart attack, is illustrated by the solid line in FIG. VI. Note that when using the conventional administration, unnecessarily high dosages of the drug are present in patients during the evening hours, when patients are least likely to experience a heart attack, and during the morning hours when most heart attacks occur, the dosage is lower than may be required.

An exemplary plasma profile for the same drug is set forth using uneven dosing, with two thirds of the total daily dosage administered at 7 am and one third of the total daily dosage administered at 11 pm, as illustrated by the dashed line in FIG. VI. Note that the uneven dosing will result in relatively high levels of the drug in the patient during the morning hours, when the patient is most vulnerable to a heart attack, and relatively low levels of the drug during the evening when the patient is least vulnerable to a heart attack.

### Example VII

The plasma profile for verapamil, when administered in a conventional form, QD at 11 pm, for the treatment and prevention of hypertension, is illustrated by the solid line in FIG. VII. Note that when using the conventional administration, sub-therapeutic levels of the drug are present in patients during a large portion of the day.

An exemplary plasma profile for the same drug is set forth using uneven dosing, with two thirds of the total daily dosage administered at 7 am and one third of the total daily dosage administered at 11 pm, as illustrated by the dashed line in FIG. VII. Note that the uneven dosing will result in more even levels of the drug throughout the day.

### Example VIII

The plasma profile for cimetidine, when administered in a conventional form, 300 mg at 7 am and 300 mg at 11 pm, for the prevention of Gastroesophageal Reflux Disease (GERD), is illustrated by the solid line in FIG. VIII. Note that when using the conventional administration, unnecessarily high dosages of the drug are present in patients during the morning hours, when patients are least likely to experience symptoms of GERD.

An exemplary plasma profile for the same drug is set forth using uneven dosing, 200 mg at 3:00 pm and 400 mg at 11 pm, as illustrated by the dashed line in FIG. VIII. Note that the uneven dosing will result in relatively low, yet adequate levels of the drug in the patient during the morning hours, when the patient is least vulnerable to the symptoms of GERD, and relatively high levels of the drug during the night when the patient is most vulnerable to the symptoms of GERD.

### Example IX

The plasma profile for cimetidine, when administered in a conventional form, 300 mg at 7 am and 300 mg at 11 pm, for the treatment of gastric ulcers, is illustrated by the solid line in FIG. IX. Note that when using the conventional administration, relatively low dosages of the drug are present in patients during the morning hours, when patients are most likely to experience symptoms associated with gastric ulcers.

An exemplary plasma profile for the same drug is set forth using uneven dosing, 200 mg at 7 am and 400 mg at 11 pm, as illustrated by the dashed line in FIG. IX. Note that the uneven dosing will result in relatively high levels of the drug in the patient during the morning hours, when the patient is most vulnerable to the symptoms associated with gastric ulcers, and relatively low levels of the drug during the night when the patient is least vulnerable to symptoms associated with gastric ulcers.

### Example X

The plasma profile for the diuretic chlorothiazide sodium, when administered in a conventional form, 25 mg at 7 am and 25 mg at 7 pm, for the treatment of hypertension, is illustrated by the solid line in FIG. X. Note that when using the conventional administration, relatively low dosages of the drug are present in patients during the daylight hours, when patients are most vulnerable to hypertension. Further, when using conventional administration, unnecessarily high dosages of the drug are present in patients during night when the patient is less vulnerable to hypertension and when the production of excess urine caused by the drug will disrupt sleep and cause the greatest degree of discomfort and inconvenience.

An exemplary plasma profile for the same drug is set forth using uneven dosing, 42 mg at 7 am and 8 mg at 5 pm, as illustrated by the dashed line in FIG. X. Note that the uneven dosing will result in relatively high levels of the drug in the patient during the daylight hours, when the patient is most vulnerable to hypertension, and relatively low levels of the drug during the night when the patient is least vulnerable to hypertension and most vulnerable to disruption of sleep and discomfort caused by the production of excessive urine.

### Example XI

Doxazosin, available under the trade name Cardura®, manufactured by Pfizer, Inc., located in New York, N.Y., is an alpha 1-adrenoceptor antagonist indicated for the treatment of hypertension. The conventional dosage regimen for doxazosin is 1-8 mg administered once a day, morning or evening, for benign prostatic hyperplasia or 1-16 mg, morning or evening, for hypertension. The Physicians' Desk Reference, 2368 (53^{rd} Ed., 1999).

In accordance with the present inventive subject matter, a drug delivery regimen is formulated by factoring into consideration various known physiological variables. In this instance, one finds that the majority of ischemic events occur between 6 am and noon. Further, one determines that the peak onset of action of doxazosin occurs within 2-4 hours of oral dosing. Moreover, available studies comparing the morning dosing of doxazosin to the evening dosing of doxazosin provide the following data:

Pharmacokinetic Study Design

24 male volunteers with nocturia (mean age 52 years and mean weight 74.1 kg) were randomized into an open-label, two-way, cross-over study with a seven day placebo washout period between 15 day treatment phases. During each treatment phase, volunteers took a daily dose of 1 mg of doxazosin for 10 days followed by a daily dose of 2 mg of doxazosin for 5 days. The two treatment phases were differentiated by the time of dosing, either 8 am or 8 pm.

TABLE II
Summary of Pk Parameters (mean values)

| Pharmacokinetic | Doxazosin | Doxazosin |
|---|---|---|
| Study (N = 24) | morning dosing | evening dosing |
| AUC_{0-24 h} (ng.h/ml) | 227.90 | 253.66 |
| Cₘₐₓ (ng/ml) | 16.98 | 15.76 |
| Tₘₐₓ (h) | 3.46 | 5.60 |
| CL/F | 2.21 | 1.97 |
| T_{1/2} (h) | 19.52 | 18.77 |

Clinical Study Design

323 male patients with clinical of BPH were randomized to receive either doxazosin or placebo in a double-blind, prospective, multi center study. Twice as many patients were randomized to doxazosin as to placebo, and within each treatment group patients were further randomized, in equal numbers, to receive study treatment at 8 am or 8 pm. The primary efficacy parameters investigated were International Prostate Symptom Score (I-PSS) and maximum urinary flow rate (Qmax), both measured at screening, after the 2 week placebo run-in period (baseline) and at 6, 12 and 24 weeks (endpoint) subsequent to baseline. After run-in, patients received either placebo or doxazosin daily for 12 weeks (1 mg for 2 weeks, then to 2 mg for 10 weeks). If an improvement in BPH symptoms (>30% in I-PSS or >3 ml/s increase in Qₘₐₓ) was not observed at his time, the dose of doxazosin was increased to 4 mg daily for the final 12 weeks of the 24-week study.

TABLE III
Clinical Efficacy Parameters (mean values)

| Clinical | | | | |
|---|---|---|---|---|
| Study | Doxazosin | Doxazosin | Placebo | Placebo |
| (N = 323) | morning | evening | morning | evening |
| I-PSS | | | | |
| Baseline | 18.5 | 18.2 | 18.6 | 18.6 |
| Endpoint | 11.0 | 11.7 | 12.0 | 13.0 |
| Mean Change | −6.9 | −6.7 | −3.9 | −5.1 |
| Responders | 57.4 | 62.6 | 42.6 | 44.2 |
| Qₘₐₓ (ml/s) | | | | |
| Baseline | 10.17 | 10.49 | 9.33 | 9.80 |
| Endpoint | 11.84 | 12.30 | 9.76 | 9.93 |
| Mean Change | 1.51 | 1.94 | 0.06 | −0.50 |
| Responders | 29.7 | 28.4 | 16.7 | 13.0 |

Analyzed by ITT and ANOVA

Study Results

Results from the Pk show that the AUC₀₋₂₄ₕ was greater and the Tₘₐₓ longer with evening doses versus morning doses. The clinical study results were more variable. However, the doxazosin significantly differed from placebo at 6 weeks and endpoint. Evening dosage groups showed a greater mean change in Qₘₐₓ (uroflow). In regard to safety, there were no serious adverse events, but hypotension did occur more often in the morning dose group (20.8%) as compared to evening (12.5%), in the cross-over Pk study.

Therefore, in accordance with the present inventive subject matter, an optimal drug delivery regimen is theorized with the smaller portion, 30%, administered on rising (6 am-8 am) and the larger dose administered with dinner (6 pm-8 pm) based upon the available information on doxazosin. Thus, for example, without limitation, the drug delivery regimen for a 1 mg total daily dosage would be a morning dose of 0.3 mg and an evening dose of 0.7 mg. This regimen can be further adjusted in time and dose as. more detailed PK data is analyzed and modeled.

It would be anticipated that the drug delivery regimen in accordance with the present inventive subject matter would provide an optimized therapeutic effect relative to conventional dosing. In particular, the present drug delivery regimen would provide more stable and prolonged drug blood levels over the night, thus reducing the risk of hypotension that may occur upon arising during the night (nocturia). Further, the present drug delivery regimen would allow reduced side effects, in particular, greater emptying of the bladder prior to bedtime.

### Example XII

Alendronate, available under the trade name Fosamax®, manufactured by Merck & Co., located in West Point, Pa., is indicated for the treatment of osteoporotic problems, especially postmenopausal osteoporosis. The conventional dosage regimen for alendronate is 5-10 mg administered once a day to treat osteoporosis and 40 mg administered once a day to treat Paget's disease. The Physicians' Desk Reference, 1795 (53^{rd} Ed., 1999).

In accordance with the present inventive subject matter, a drug delivery regimen is formulated by factoring into consideration various known physiological variables. In this instance, one finds that bone resorption shows a circadian rhythm, occurring during sleep and/or recumbency. Therefore, therapeutic regimens of alendronate target effective action during sleep. Alendronate causes esophageal irritation and reflux which can progress to ulceration. Further, oral dose forms are not easily absorbed in the gut, but too much alenodronate causes osteomalacia.

Therefore, in accordance with the inventive subject matter, the dosage would be 5 mg per day for prophylaxis and 10-40 mg per day for treatment of osteoporosis. 60-75% of the total dose is taken 2 hours after the evening meal and at least one hour-before bed. The remaining 40-25% of the dose is taken in the morning upon rising and before eating breakfast. This morning dose should cover the tail end of the recumbent bone resorption period of the night before, and should prevent overdose by splitting the dose into two parts. The reduced evening dose reduces the chance of side effects such as acid reflux and osteomalacia.

The drug delivery regimen in accordance with the present inventive subject matter would provide an optimized therapeutic effect relative to conventional dosing. In particular, the present drug delivery system provides more effective dosing by providing the maximum drug over night when bone reabsorption activity is highest. Further, the present drug delivery system is designed to provide prolonged drug coverage through by administering a dose in the morning as well as the evening. Finally, the present drug delivery system would reduce side effects, such as acid reflux and osteomalacia.

### Example XIII

Cisapride monohydrate, available under the trade name Propulsid®, manufactured by Janssen, located in Titusville, New Jersey, is an H₂ antagonist indicated for the treatment of gastroesophageal reflux disease (GERD). The conventional dosage regimen of cisapride monohydrate is 10-20 mg four times a day. The Physicians' Desk Reference, 1430 (53^{rd} Ed., 1999). This frequent dosing reduces patient compliance.

In accordance with the present inventive subject matter, a drug delivery regimen is formulated by factoring into consideration various known physiological variables. In this instance, one finds that gastric activity follows a circadian rhythm such that there is more acid production at certain times than at other times. GERD is primarily a nocturnal disorder, and the greatest acid load occurs after the ingestion of the evening meal and through the night.

Therefore, in accordance with the present inventive subject matter, an optimal drug delivery regimen is theorized based on the acid load currently in the stomach. The majority of the dose would be administered in the evening.

The drug delivery regimen in accordance with the present inventive subject matter would provide an optimized therapeutic effect relative to conventional dosing. In particular, the present drug regimen would increase patient compliance due to less frequent dosing and the patient not having to dose with every meal. Further, the evening dose would provide a stable and prolonged drug blood level through the night.

### Example XIV

AZT, available under the trade name Zidovudine,® manufactured by Glaxo Wellcome, located in Research Triangle Park, N.C., is a deoxynucleoside indicated for the treatment of HIV infection. The conventional dosage regimen for AZT is 500-600 mg each day (maximum of 1500 mg) by mouth.

In accordance with the present inventive subject matter, a drug delivery regimen is formulated by factoring into consideration various known physiological variables. AZT dosing may be adjusted according to immune cell patterns. Accordingly, a higher dose of AZT should be given at night, when circulating immune cell levels are low, and bone marrow toxicity is lowest. AZT should be kept at lower levels during the afternoon, when bone marrow proliferation is at peak levels. Available studies comparing AZT toxicity levels with bone marrow suppression provide the following data:

Animal Studies

AZT toxicity to the bone marrow (BM) is its major hindrance to use in clinical application for the treatment of AIDS. There is a mathematical model which can predict that cytotoxicity to the host can be reduced when the frequency of drug administration is an integer multiple of the target cell average cycle time (circa 7 hours in murine bone cells). In vivo experiments in mice show that a 7 hour frequency of AZT administration is significantly less toxic than other frequencies when peripheral blood parameters and the proportion of bone marrow cells arrested at the S-phase gate of the DNA content distribution are considered.

AZT's major drug-related toxicity is bone marrow suppression, which limits the dose of AZT that can be used. It is essential that AZT be phosphorylated for its antiviral effect. Thymidine kinase (TK), the initial enzyme in AZT anabolism, follows a circadian pattern in rat bone marrow. AZT-related toxic effects, including bone marrow toxicity, differ significantly among the treatment groups, depending on the time of AZT administration. The least toxicity was observed in rats receiving AZT at 4 pm (10 hours after light onset [HALO], in the late sleep time span) and the greatest toxicity was observed in those injected at 4 am (22 HALO, in the late activity time span).

Human Studies

AZT is associated with unacceptable levels of bone marrow suppression, and ddC can cause painful peripheral neuropathy. The different toxicity profiles of these 2 drugs provide the rational for testing them in alternating dosing combinations in an attempt to retain the antiretroviral activity of each against HIV, while reducing the toxicities of both. A preliminary trial showed that 200 mg AZT given orally every 4 hours for 7 day periods, alternating with ddC at 0.03 mg/kg body weight orally every 4 hours for 7 day periods is a promising treatment regimen. Alternating regimens of AZT and ddC not only might decrease toxicity associated with the 2 drugs, but may prove to be more efficious than AZT alone.

A study comparing doses of 400 mg each day to 800 mg each day was conducted enrolling patients with HIV infection. The effective rate if AZT on CD4+ lymphocyte counts was similar for both groups, but the duration of the effect of AZT was significantly longer in the 400 mg group. In the 800 mg group, adverse reaction were more frequently observed, and AZT was withdrawn or the dose was reduced more frequently. These results suggest that AZT at a dose of 400 mg each day is less toxic, and more beneficial for long term treatment.

Therefore, in accordance with the present inventive subject matter, an optimal drug delivery regimen is theorized with the lowest blood level in the early afternoon and the highest level at night. Thus, for example, without limitation, the drug delivery regimen for 550 mg would be:

After Breakfast Dose (0600-0900 hours) 150 mg ˜8 hour interval to late afternoon dose

Before Dinner Dose (1500-1800 hour) 250 mg ˜6 hour interval to bedtime dose

Bedtime Dose (2100-2400) 150 mg ˜9 hour interval to morning dose.

The drug delivery regimen in accordance with the present inventive subject matter would provide an optimized therapeutic effect relative to conventional dosing. In particular, the present drug regimen keeps the blood level of AZT lowest at the time bone marrow proliferation is most sensitive to cytotoxic drugs. Further, the highest doses are administered when levels of circulating antiviral cells are lowest and bone marrow toxicity is shown to be lowest.

### Example XV

Carboplatin, available under the trade name Paraplatin®, manufactured by Bristol-Myers Squibb, located in New York, N.Y., is indicated for the treatment of cancer. The conventional dosage regimen of carboplatin is 300-600 mg/m² I.V. every 4 weeks. The Physicians' Desk Reference, 789 (53^{rd} Ed., 1999).

In accordance with the present inventive subject matter, a drug delivery regimen is formulated by factoring into consideration various known physiological variables. In this instance, one finds that the time of day anti-cancer drugs are given has an effect both on the effectiveness and the toxicity of the drugs. The drug efficacy and toxicity are inversely related, and evening has been found to be the point of highest efficacy and lowest toxicity. The lowest marrow toxicity occurs when the drug is received at the beginning of the sleep phase. In the mouse model, the longest mean survival time and the lowest marrow toxicity occurred in the group which received the drug at the beginning of the sleep phase.

Therefore, in accordance with the inventive subject matter, an optimal drug regimen is theorized using circadian rhythms. Circadian biorhythms can be determined before drug administration and used to determine the optimal time of dose. The data indicates evening dosage is optimal.

The drug delivery regimen in accordance with the present inventive subject matter would provide an optimized therapeutic effect relative to conventional dosing. In particular, the present drug regimen is administered when the body is least susceptible to cytotoxic drugs and when levels of the bodys' own antiviral defense system is weakest.

### Example XVI

Fluticasone propionate, available under the trade name Flovent® or Flonase®, manufactured by Glaxo Wellcome, located in Research Triangle Park, N.C., is an inhaled steroid indicated for the treatment of asthma. The conventional dosage regimen of fluticasone propionate is 100-200 mcg/day. The Physicians' Desk Reference, 1122-24 (^{53rd} Ed., 1999).

In accordance with the present inventive subject matter, a drug delivery regimen is formulated by factoring into consideration various known physiological variables. In this instance, one finds that a variety of circadian rhythms play a role in the causes of nocturnal asthma, such as those of platelet function, and circulating immune cells.

Therefore, in accordance with the present inventive subject matter, an optimal drug delivery regimen is theorized to make use of these rhythms. Thus, for example, without limitation, the optimal dosage time appears to be between 3 pm and 5:30 pm to minimize cortisol suppression. Therefore, in accordance with the inventive subject matter, fluticasone propionate should be administered once during a twenty four hour period with a dosage range of 250-1000 mcg during the period between 3:00 pm and 5:30 pm.

The drug delivery regimen in accordance with the present inventive subject matter would provide an optimized therapeutic effect relative to conventional dosing. In particular, the present drug regimen minimizes side effects, such as the suppression of cortisol excretion, as well as immunosupression and thrush.

### Example XVII

HMG CoA reductase inhibitors, a subclass of lipid lowering agents, are indicated in the treatment of high cholesterol.

In accordance with the present inventive subject matter, a drug delivery regimen is formulated by factoring into consideration various known physiological variables. In this instance, one finds that circadian rhythms for total cholesterol, as well as HDL-cholesterol, beta-lipoproteins and triglycerides, can be seen in elderly persons. In patient groups with high cholesterol, the highest concentration of the lipid fractions was seen at 2-4 pm. Further, cholesterol is seen to decrease late at night and very early in the morning.

Therefore, in accordance with the present inventive subject matter, an optimal drug delivery regimen is theorized to make use of these rhythms. Thus, for example, without limitation, the optimal dosage time is 2-4 pm.

### Example XVIII

Enoxaparin, available under the trade name Lovenox®, manufactured by Rhone-Poulenc-Rorer, located in Collegeville, Pa., is indicated in the treatment of DVT and pulmonary emboli following major orthopedic surgery, as well as an anticoagulant following myocardial infarction, angina, coronary artery disease treatment and angioplasty. The conventional daily dosage regimen of enoxaparin is 30 mg per 12 hours or 40 mg per day. The Physicians' Desk Reference, 2591 (^{53rd} Ed., 1999).

In accordance with the present inventive subject matter, a drug delivery regimen is formulated by factoring into consideration various known physiological variables. In this instance, one finds that cardiac disorders show an increased occurrence during the time surrounding awakening. The highest points occur about 90 minutes after first assuming an upright posture for the day.

Therefore, in accordance with the present inventive subject matter, an optimal drug delivery regimen is theorized with the larger dosage given at night. If bleeding occurs from the peak levels of the night dose (around 4 am), then a larger morning dose may be provided. For example, without limitation, the drug delivery regimen for 60-65 mg total daily dosage would be one 40 mg dose at bedtime (8 pm-midnight) and a 20-25 mg dose upon awakening.

The drug delivery regimen in accordance with the present inventive subject matter would provide an optimized therapeutic effect relative to conventional dosing. In particular, the present drug regimen provides anticoagualtion coverage for the vulnerable period before and during awakening.

### Example XIX

Premarin®, manufactured by Wyeth-Ayerst Laboratories, located in Philadelphia, Pa., is a conjugated estrogen indicated in the treatment of vasomotor symptoms associated with menopause, atrophic vaginitis and osteoporosis. The conventional dosage regimen for Premarin® is 1.25 mg each day. The Physicians' Desk Reference, 3370 (^{53rd} Ed., 1999).

In accordance with the present inventive subject matter, a drug delivery regimen is formulated by factoring into consideration various known physiological variables. In this instance, one finds that many estrogen effects are used in treatment of other diseases, and the estrogens should be dosed in synchronization with patterns of efficacy and safety for these diseases.

Estrogen Effects on Lipids

The effects of conjugated estrogens administered at 8 am and 8 pm, on serum lipoproteins, were studies in post-menopausal women. Results showed only decreased levels of lipoprotein in the 8 pm group. The results seem to be dependant on the circadian rhythm of the hepatic responsiveness to estrogens, whose expression is higher in the evening hours.

Estroqen Effects on Adverse Hypercoagulation

The onset of acute atherothrombotic events (acute myocardial infarction, angina and ischemic stroke) exhibit a circadian pattern that parallels the diurnal pattern of endogenous fibrolytic activity. Platelet aggregation peaks at the time of awakening, and increases between 9 am and 11 am (2 hours after assuming the upright posture). In an investigation of coagulation system activation following estrogen treatment in healthy post-menopausal women who received conjugated estrogens at 0.625 and 1.25 mg each day versus placebo for 3 months in a randomized, cross-over protocol. Blood samples were obtained on 2 consecutive days at the end of each treatment period for immunoassays of F1+2 and fibrinopeptide A (FPA), markers of factor Xa action of prothrombin and thrombin action on fibrogin respectively. Treatment with estrogens at a dose of 0.625 or 1.25 mg resulted in significant increases in mean F1+2 levels (40 and 98%), and in mean FPA levels (37 and 71%). The measurements of F1+2 were significantly higher in women receiving 1.25 mg of estrogen than in those receiving 0.625 mg. Hence, low doses of oral estrogens (</=1.25 mg each day) may increase the amount of thrombin generated in vivo.

Estrogen Effects on Vasomotor Effects

In a study evaluating short-term endothelium-dependant vascular effects of intravenously conjugated estrogen at 2.5 and 5 mg of conjugated estrogen or placebo in random order in a double-blind study design. The vascular reactivity of the brachial artery was studied before and 30 minutes after the intravenous administration of the study drug. Reactive hyperemia was used to study the flow-mediated vasodilation. Serum estradiol increased dose dependently 5 minutes after the conjugated estrogen infusion. Conjugated estrogen at a dose of 2.5 mg caused an increase in flow-mediated vasodilation from 1.8 at baseline to 5.4 after infusion, whereas 5 mg caused an increase from 1.9 at baseline to 7.0 after infusion. Intravenous injection of conjugated estrogen significantly improves the peripheral vascular reactivity in postmenopausal women.

Estrogen Effects on Bone Mass

Bone reabsorption shows a circadian rhythm in human subjects. 24 patients with established osteoporosis and with ten or more years of menopause were treated with conjugated estrogen, progesterone and calcium. Treated women received 0.625 mg each day of conjugated estrogen from day 1 to 25 of each cycle, plus 500-1000 mg each day of calcium, for 1 year (12 cycles). The control group only received calcium. Estrogen treatment was associated with increased bone mineral density at the spine and the trocanter. The control group did not present any statistical change after 1 year in any site studied. This data supports the theory that women with ten or more years of menopause respond to estrogen replacement therapy in a way similar to younger women in the early phases of menopause.

Therefore, in accordance with the present inventive subject matter, an optimal drug delivery regimen is theorized with the larger dose given at bedtime. For example, without limitation, the drug delivery regimen for 1.0 mg would be 0.75 mg with the evening meal or at bedtime (65-75% of total) and 0.25 mg upon rising or after the morning meal (25-35% of total).

The drug delivery regimen in accordance with the present inventive subject matter would provide an optimized therapeutic effect relative to conventional dosing. In particular, the present drug regimen prevents provides a steady level of estrogen through the night by making the evening dose of estrogen larger than the morning dose. This stable night level of estrogen is assists in bone reabsorption, which is worst during recumbency. Further, an enhanced coagulation state by taking the morning estrogen dose after assuming an upright posture. Finally, in splitting the dose, rather than administering one large dose, the severity of hot flashed caused by vasoconstriction and vasodilation is reduced.

The invention being thus described, it will be apparent that the same may be varied in many ways. Such variations are not to be regarded as a departure from the spirit and scope of the invention, and all such modifications are intended to be within the scope of the appended claims.

We claim:

1. A drug delivery regimen, which comprises:
an active therapeutic substance(s) selected from the group consisting of anti-hypertensive agents, osteoporotic agents, GERD agents, anti-viral agents, anti-neoplastic agents, inhaled steroids, lipid lowering agents, thrombolytic agents, anticoagulant agents, fibrinolytic agents, anti-asthmatics, hormone replacement agents, anti-infectives, anti-diabetics, vitamins, herbal agents, minerals, fatty acids, electrolytes and combinations thereof administered multiple times during at least one 24 hour period of time to provide effective therapeutic levels of the active therapeutic substance(s) at a site or sites of action in an animal over said period, wherein each individual dose is independently adjusted to be administered to optimize levels of the active therapeutic substance(s) at the site or sites of action for maximum efficacy, and wherein the dose amount at each administration is independently characterized by the formula TD(t)=CD(t)+RD(t), where t is the time at which the dose is to be administered, TD (therapeutic dose) is the therapeutically effective dose at time (t), CD (current dose) is the dose to be administered at time (t), and RD (residual dose) is the amount of active therapeutic substance(s) remaining from the previous dose administration.

2. The drug delivery regimen of claim 1, wherein the drug delivery regimen agent is selected from the group consisting of calcium channel blockers, ACE inhibitors, angiotensin II receptor antagonists, beta-adrenoceptor antagonists, alpha 1-adrenoceptor antagonists, alpha 2-adrenoceptor antagonists, diuretics and combinations thereof.

3. The drug delivery regimen of claim 2, wherein the calcium channel blocker is nifedipine, verapamil, nicardipine, diltiazem, isradipine, amlodipine, felodipine, nifedipine, bepridil and combinations thereof.

4. The drug delivery regimen of claim 2, wherein the ACE inhibitor is quinapril, ramipril, captopril, benazepril, fosinopril, lisinopril, moexipril, enalapril and combinations thereof.

5. The drug delivery regimen of claim 2, wherein the angiotensin II receptor antagonist is losartan.

6. The drug delivery regimen of claim 2, wherein the beta adrenoceptor antagonist is sotalol, timolol, esmolol, carteolol, propanolol, betaxolol, penbutolol, metoprolol, labetalol, acebutolol, atenolol, bisoprolol and combinations thereof.

7. The drug delivery regimen of claim 2, wherein the alpha 1-adrenoceptor antagonist is doxazosin, phenoxybenzamine, guanethidine, guanadrel, terazosin, prazosin and combinations thereof.

8. The drug delivery regimen of claim 2, wherein the alpha 2-adrenoceptor agonist is methyldopa, clonidine, guanfacine and combinations thereof.

9. The drug delivery regimen of claim 2, wherein the diuretic is selected from the group consisting of carbonic anhydrase inhibitors, loop diuretics, thiazides, potassium sparing diuretics and combinations thereof.

10. The drug delivery regimen of claim 1, wherein the osteoporotic agent is alendronate, etidronate, pamidronate, clodronate, tiludronate, residronate, ibandronate and combinations thereof.

11. The drug delivery regimen of claim 1, wherein the GERD agent is oral GI prokinetic agents, agents active against H. Pylori, proton pump inhibitors, H₂ histamine receptor antagonists, antacids and combinations thereof.

12. The drug delivery regimen of claim 11, wherein the oral GI prokinetic agent is cisapride monohydrate, metoclopramide and combinations thereof.

13. The drug delivery regimen of claim 11, wherein the agent active against H. Pylori is clarithromycin, tetracycline, amoxicillin, bismuth, metronidazole and combinations thereof.

14. The drug delivery regimen of claim 11, wherein the proton pump inhibitor is omeprazole, lansoprazole and combinations thereof.

15. The drug delivery regimen of claim 11, wherein the H₂ histamine receptor antagonist is cimetadine, famotidine, nizatidine, ranitidine, roxatidine and combinations thereof.

16. The drug delivery regimen of claim 1, wherein the anti-viral agent is nucleoside analogs, protease inhibitors and combinations thereof.

17. The drug delivery regimen of claim 16, wherein the nucleoside analog is zidovudine, azidothymidine, didanosine, zalcitabine, stavudine, lamivudine and combinations thereof.

18. The drug delivery regimen of claim 16, wherein the protease inhibitor is saquinavir mesylate, ritonavir, indinavir and combinations thereof.

19. The drug delivery regimen of claim 1, wherein the anti-neoplastic agent is selected from the group consisting of cytoxic agents, anti-metabolites, platinum-containing compounds, antibiotic derivatives, fluoropyrimidines, nitrosoureas, vinca alkaloids, nitrogen mustard derivatives, adjuvant biological response modifiers and combinations thereof.

20. The drug delivery regimen of claim 19, wherein the cytoxic agent is paclitaxel, cyclophosphamide, teniposide and combinations thereof.

21. The drug delivery regimen of claim 19, wherein the anti-metabolite is methotrexate.

22. The drug delivery regimen of claim 19, wherein the platinum-containing compound is cisplatin (cis-diaminedichlororoplatinum), carboplatin, oxaliplatin and combinations thereof.

23. The drug delivery regimen of claim 19, wherein the antibiotic derivative is adriamycin, bleomycin, dactinomycin, daunorubicin, doxorubicin, indarubicin, mytomycin and combinations thereof.

24. The drug delivery regimen of claim 19, wherein the fluoropyrimidine is 5-FU (5-fluorouracil), FudR (5-fluoro-2′-deoxyuridine), Ara-C (arabinosylcytosine) and combinations thereof.

25. The drug delivery regimen of claim 19, wherein the nitrosourea is BCNU (carmustine), streptozocin and combinations thereof.

26. The drug delivery regimen of claim 19, wherein the vinca alkaloid is vinblastine, vincristine and combinations thereof.

27. The drug delivery regimen of claim 19, wherein the nitrogen mustard derivative is thiotepa.

28. The drug delivery regimen of claim 19, wherein the adjuvant biological response modifier is selected from the group consisting of alpha-interferon, TNF (tumor necrosis factor), EPO (erythropoietin), rhG-CSF (recombinant human granulocyte colony-stimulating factor), IL-1 (interleukin-1), IL-2 (interleukin-2), monoclonal antibodies to tumor and immunologic targets and combinations thereof.

29. The drug delivery regimen of claim 1, wherein the inhaled steroid is beclomethasone dipropionate, budesonide, flunisolide, fluticasone propionate, mometasone furoate, triamcinolone acetonide and combinations thereof.

30. The drug delivery regimen of claim 1, wherein the lipid lowering agent is nicotinic acid, HMG CoA reductase inhibitors, bile sequestration agents, fibric acid derivatives and combinations thereof.

31. The drug delivery regimen of claim 30, wherein the HMG CoA reductase inhibitor is atorvastatin, cerivastatin, fluvastatin, lovastatin, pravastatin, simvastatin and combinations thereof.

32. The drug delivery regimen of claim 30, wherein the bile sequestration agent is colestipol, cholestyramine and combinations thereof.

33. The drug delivery regimen of claim 30, wherein the fibric acid derivative is clofibrate, gemfibrozil and combinations thereof.

34. The drug delivery regimen of claim 1, wherein the thrombolytic, anticoagulant, fibrinolytic agent is selected from the group consisting of heparin-like agents, clot buster agents, aspirin-like agents, platelet glycoprotein IIb, IIIa receptor antagonists and combinations thereof.

35. The drug delivery regimen of claim 34, wherein the heparin-like agent is selected from the group consisting of enoxaparin, dalteparin, refludan and combinations thereof.

36. The drug delivery regimen of claim 34, wherein the clot buster agent is streptokinases, alteplase (TPA) and combinations thereof.

37. The drug delivery regimen of claim 34, wherein the aspirin-like agent is a thromboxane inhibitor.

38. The drug delivery regimen of claim 34, wherein the platelet glycoprotein IIb, IIIa receptor antagonist is tirofiban, eptifibatide, abciximab and combinations thereof.

39. The drug delivery regimen of claim 1, wherein the vitamin thiamine, niacinamide, pyridoxine, ascorbic acid, riboflavin, folic acid, vitamin A, vitamin E, vitamin D3, cyanocobalamin, biotin, pantothenic acid, derivatives thereof, and combinations thereof.

40. The drug delivery regimen of claim 1, wherein the herbal agent is black cohosh, licorice, false unicorn, siberian ginseng, sarsaparilla, squaw vine, blessed thistle, peppermint, spearmint, red raspberry, St. Johnswort, ginger, kola, hops, valerian, derivatives thereof and combinations thereof.

41. The drug delivery regimen of claim 1, wherein the fatty acid is selected from the group consisting of linoleic acid, linolenic acid, docosahexaenoic acid, arachidonic acid, eicosahexaneoic acid, omega-3 fatty acid, omega-2 fatty acid, derivatives thereof and combinations thereof.

42. The drug delivery regimen of claim 1, wherein the mineral is selected from the group consisting of copper, zinc, iodide, magnesium, chromium, molybdenum, sodium, calcium, iron, fluoride, phosphorus, manganese, potassium, boron, selenium, bioflavonoid, phosphate, derivatives thereof and combinations thereof.

43. The drug delivery regimen of claim 1, wherein the electrolyte is selected from the group consisting of potassium, magnesium, sodium, calcium, derivatives thereof and combinations thereof.

44. The drug delivery regimen of claim 1, wherein the active therapeutic substance(s) is administered to increase efficacy.

45. The drug delivery regimen of claim 1, wherein the active therapeutic substance(s) is administered to reduce the total dosage administered per day while maintaining equivalent efficacy.

46. The drug delivery regimen of claim 1, wherein the active therapeutic substance(s) is administered to minimize incidents of side effects.

47. The drug delivery regimen of claim 1, wherein the active therapeutic substance(s) is administered to improve patient compliance with the drug delivery regimen.

48. The drug delivery regimen of claim 1, wherein the active therapeutic substance(s) is administered to improve convenience of administration.

49. The drug delivery regimen of claim 1, wherein the active therapeutic substance(s) is administered at least once and may be administered as immediate release, sustained release, controlled release, delayed release, timed release, extended release, or any combination thereof.

50. The drug delivery regimen of claim 5, wherein the active therapeutic substance(s) is administered by pulsatile delivery of the active therapeutic substance(s).

51. The drug delivery regimen of claim 1, wherein the active therapeutic substance(s) is administered in one or more dosage forms independently selected from the group consisting of chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods, by a vaporizer and combinations thereof.

52. The drug delivery regimen of claim 1, wherein the active therapeutic substance(s) is administered in two or more dosage forms independently selected from the group consisting of tablet, multi-layer tablet, capsule, or caplet.

53. The drug delivery regimen of claim 52, wherein the multi-layer tablet is composed of an extended-release layer and an immediate release layer.

54. The drug delivery regimen of claim 52, wherein the dosage form is coated for ease of administration, coated for delayed release or enteric coated to reduce gastric irritation.

55. The drug delivery regimen of claim 52, wherein the dosage form is enteric coated and compressed into a tablet or filled into hard or soft gelatin capsules.

56. The drug delivery regimen of claim 1, wherein the active therapeutic substance(s) is administered in uneven doses.

57. The drug delivery regimen of claim 1, wherein the active therapeutic substance(s) is administered at uneven time intervals over the course of the 24 hour period.

58. The drug delivery regimen of claim 1, wherein an AM dose and a PM dose are administered, and wherein the AM dose is larger or smaller than the PM dose.

59. The drug delivery regimen of claim 1, wherein an AM dose and a PM dose are administered, and wherein the AM dose has a higher or lower amount of a water-soluble active therapeutic substance(s) present than that present in the PM dose.

60. The drug delivery regimen of claim 1, wherein an AM dose and a PM dose are administered, and wherein the AM dosage has a higher or lower amount of a non water-soluble drug present than that present in the PM dosage.

61. The drug delivery regimen of claim 1, wherein the dosage is adjusted for subsequent 24 hour periods of time.

62. The drug delivery regimen of claim 1, wherein the active therapeutic substance(s) is substituted for another active therapeutic substance(s).

63. The drug delivery regimen of claim 1, wherein two PM doses are administered, and wherein the first PM dose is administered immediately after dinner and the second PM dose is administered immediately prior to bedtime.

64. A drug delivery regimen, which comprises:
at least two dose of an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a vitamin, an herbal agent, a mineral, a fatty acid, an electrolyte and combinations thereof administered during at least one 24 hour period of time to provide effective therapeutic levels of the active therapeutic substance(s) at a site or sites of action in an animal over said period, wherein the active therapeutic substance(s) is administered in uneven doses and over varying time intervals, and wherein the uneven doses and the varying time intervals are selected to optimize levels of the active therapeutic substance(s) at the site or sites of action for maximum efficacy.

65. The drug delivery regimen of claim 64, wherein the anti-hypertensive agent is selected from the group consisting of a calcium channel blocker, an ACE inhibitor, an angiotensin II receptor antagonist, a beta-adrenoceptor antagonist, an alpha 1-adrenoceptor antagonists, an alpha 2-adrenoceptor antagonist, a diuretic and combinations thereof.

66. The drug delivery regimen of claim 65 wherein the calcium channel blocker is selected from the group consisting of nifedipine, verapamil, nicardipine, diltiazem, isradipine, amlodipine, felodipine, nifedipine, bepridil and combinations thereof.

67. The drug delivery regimen of claim 65, wherein the ACE inhibitor is selected from the group consisting of quinapril, ramipril, captopril, benazepril, fosinopril, lisinopril, moexipril, enalapril and combinations thereof.

68. The drug delivery regimen of claim 65 wherein the angiotensin II receptor antagonist is losartan.

69. The drug delivery regimen of claim 65, wherein the beta adrenoceptor antagonist is selected from the group consisting of sotalol, timolol, esmolol, carteolol, propanolol, betaxolol, penbutolol, metoprolol, labetalol, acebutolol, atenolol, bisoprolol and combinations thereof.

70. The drug delivery regimen of claim 65, wherein the alpha 1-adrenoceptor antagonist is selected from the group consisting of doxazosin, phenoxybenzamine, guanethidine, guanadrel, terazosin, prazosin and combinations thereof.

71. The drug delivery regimen of claim 65, wherein the alpha 2-adrenoceptor agonist is selected from the group consisting of methyldopa, clonidine, guanfacine and combinations thereof.

72. The drug delivery regimen of claim 65, wherein the diuretic is selected from the group consisting of carbonic anhydrase inhibitors, loop diuretics, thiazides, potassium sparing diuretics and combinations thereof.

73. The drug delivery regimen of claim 64, wherein the osteoporotic agent is selected from the group consisting of alendronate, etidronate, pamidronate, clodronate, tiludronate, residronate, ibandronate and combinations thereof.

74. The drug delivery regimen of claim 64, wherein the GERD agent is selected from the group consisting of oral GI prokinetic agents, agents active against H. Pylori, proton pump inhibitors, H₂ histamine receptor antagonists, antacids and combinations thereof.

75. The drug delivery regimen of claim 74, wherein the oral GI prokinetic agent is selected from the group consisting of cisapride monohydrate, metoclopramide and combinations thereof.

76. The drug delivery regimen of claim 74, wherein the agent active against H. Pylori is selected from the group consisting of clarithromycin, tetracycline, amoxicillin, bismuth, metronidazole and combinations thereof.

77. The drug delivery regimen of claim 74, wherein the proton pump inhibitor is selected from the group consisting of omeprazole, lansoprazole and combinations thereof.

78. The drug delivery regimen of claim 74, wherein the H₂ histamine receptor antagonist is selected from the group consisting of cimetadine, famotidine, nizatidine, ranitidine, roxatidine and combinations thereof.

79. The drug delivery regimen of claim 64, wherein the anti-viral agent is selected from the group consisting of nucleoside analogs, protease inhibitors and combinations thereof.

80. The drug delivery regimen of claim 79, wherein the nucleoside analog is selected from the group consisting of zidovudine, azidothymidine, didanosine, zalcitabine, stavudine, lamivudine and combinations thereof.

81. The drug delivery regimen of claim 79, wherein the protease inhibitor is selected from the group consisting of saquinavir mesylate, ritonavir, indinavir and combinations thereof.

82. The drug delivery regimen of claim 64, wherein the anti-neoplastic agent is selected from the group consisting of cytoxic agents, anti-metabolites, platinum-containing compounds, antibiotic derivatives, fluoropyrimidines, nitrosoureas, vinca alkaloids, nitrogen mustard derivatives, adjuvant biological response modifiers and combinations thereof.

83. The drug delivery regimen of claim 82, wherein the cytoxic agent is selected from the group consisting of placlitaxel, cyclophosphamide, teniposide and combinations thereof.

84. The drug delivery regimen of claim 82, wherein the anti-metabolite is methotrexate.

85. The drug delivery regimen of claim 82, wherein the platinum-containing compound is selected from the group consisting of cisplatin (cis-diaminedichlororoplatinum), carboplatin, oxaliplatin and combinations thereof.

86. The drug delivery regimen of claim 82, wherein the antibiotic derivative is selected from the group consisting of adriamycin, bleomycin, dactinomycin, daunorubicin, doxorubicin, indarubicin, mytomycin and combinations thereof.

87. The drug delivery regimen of claim 82, wherein the fluoropyrimidine is selected from the group consisting of 5-FU (5-fluorouracil), FudR (5-fluoro-2′-deoxyuridine), Ara-C (arabinosylcytosine) and combinations thereof.

88. The drug delivery regimen of claim 82, wherein the nitrosourea is selected from the group consisting of BCNU (carmustine), streptozocin and combinations thereof.

89. The drug delivery regimen of claim 82, wherein the vinca alkaloid is selected from the group consisting of vinblastine, vincristine and combinations thereof.

90. The drug delivery regimen of claim 82, wherein the nitrogen mustard derivative is thiotepa.

91. The drug delivery regimen of claim 82, wherein the adjuvant biological response modifier is selected from the group consisting of alpha-interferon, TNF (tumor necrosis factor), EPO (erythropoietin), rhG-CSF (recombinant human granulocyte colony-stimulating factor), IL-1 (interleukin-1), IL-2 (interleukin-2), monoclonal antibodies to tumor and immunologic targets and combinations thereof.

92. The drug delivery regimen of claim 64, wherein the inhaled steroid is selected from the group consisting of beclomethasone dipropionate, budesonide, flunisolide, fluticasone propionate, mometasone furoate, triamcinolone acetonide and combinations thereof.

93. The drug delivery regimen of claim 64, wherein the lipid lowering agent is selected from the group consisting of nicotinic acid, HMG CoA reductase inhibitors, bile sequestration agents, fibric acid derivatives and combinations thereof.

94. The drug delivery regimen of claim 93, wherein the HMG CoA reductase inhibitor is selected from the group consisting of atorvastatin, cerivastatin, fluvastatin, lovastatin, pravastatin, simvastatin and combinations thereof.

95. The drug delivery regimen of claim 93, wherein the bile sequestration agent is selected from the group consisting of colestipol, cholestyramine and combinations thereof.

96. The drug delivery regimen of claim 93, wherein the fibric acid derivative is selected from the group consisting of clofibrate, gemfibrozil and combinations thereof.

97. The drug delivery regimen of claim 64, wherein the thrombolytic, anticoagulant, fibrinolytic agent is selected from the group consisting of heparin-like agents, clot buster agents, aspirin-like agents, platelet glycoprotein IIb, IIIa receptor antagonists and combinations thereof.

98. The drug delivery regimen of claim 97, wherein the heparin-like agent is selected from the group consisting of enoxaparin, dalteparin, refludan and combinations thereof.

99. The drug delivery regimen of claim 97, wherein the clot buster agent is selected from the group consisting of streptokinases, alteplase (TPA) and combinations thereof.

100. The drug delivery regimen of claim 97, wherein the aspirin-like agent is a thromboxane inhibitor.

101. The drug delivery regimen of claim 97, wherein the platelet glycoprotein IIb, IIIa receptor antagonist is selected from the group consisting of tirofiban, eptifibatide, abciximab and combinations thereof.

102. The drug delivery regimen of claim 64, wherein the vitamin is selected from the group consisting of thiamine, niacinamide, pyridoxine, ascorbic acid, riboflavin, folic acid, vitamin A, vitamin E, vitamin D₃, cyanocobalamin, biotin, pantothenic acid, derivatives thereof, and combinations thereof.

103. The drug delivery regimen of claim 64, wherein the herbal agent is selected from the group consisting of black cohosh, licorice, false unicorn, siberian ginseng, sarsaparilla, squaw vine, blessed thistle, peppermint, spearmint, red raspberry, St. Johnswort, ginger, kola, hops, valerian, derivatives thereof and combinations thereof.

104. The drug delivery regimen of claim 64, wherein the fatty acid is selected from the group consisting of linoleic acid, linolenic acid, docosahexaenoic acid, arachidonic acid, eicosahexaneoic acid, omega-3 fatty acid, omega-2 fatty acid, derivatives thereof and combinations thereof.

105. The drug delivery regimen of claim 64, wherein the mineral is selected from the group consisting of copper, zinc, iodide, magnesium, chromium, molybdenum, sodium, calcium, iron, fluoride, phosphorus, manganese, potassium, boron, selenium, bioflavonoid, phosphate, derivatives thereof and combinations thereof.

106. The drug delivery regimen of claim 64, wherein the electrolyte is selected from the group consisting of potassium, magnesium, sodium, calcium, derivatives thereof and combinations thereof.

107. The drug delivery regimen of claim 64, wherein the active therapeutic substance(s) is administered at least once and may be administered as immediate release, sustained release, controlled release, delayed release, timed release, extended release, or any combination thereof.

108. The drug delivery regimen of claim 107, wherein the active therapeutic substance(s) is administered by pulsatile delivery of the active therapeutic substance(s).

109. The drug delivery regimen of claim 64, wherein the active therapeutic substance(s) is administered in a dosage form independently selected from the group consisting of liquid, solution, suspension, emulsion, tablet, multi-layer tablet, capsule, soft gelatin capsule, caplet, lozenge, chewable lozenge, bead, powder, granules, dispersible granules, cachets, douche, suppository, cream, topical, inhalant, patch, particle inhalant, implant, ingestible, injectable, infusion health bar, confection, animal feed, cereal, cereal coating, food, nutritive food, functional food and combinations thereof.

110. The drug delivery regimen of claim 64, wherein the active therapeutic substance(s) is administered in a dosage form independently selected from the group consisting of tablet, multi-layer tablet, capsule, or caplet.

111. The drug delivery regimen of claim 110, wherein the multi-layer tablet is composed of an extended-release layer and an immediate release layer.

112. The drug delivery regimen of claim 110, wherein the dosage form is coated for ease of administration, coated. for delayed release or enteric coated to reduce gastric irritation.

113. The drug delivery regimen of claim 110, wherein the dosage form is enteric coated and compressed into a tablet or filled into hard or soft gelatin capsules.

114. The drug delivery regimen of claim 64, wherein the active therapeutic substance(s) is administered in uneven doses.

115. The drug delivery regimen of claim 64, wherein the active therapeutic substance(s) is administered at uneven time intervals over the course of the 24 hour period.

116. The drug delivery regimen of claim 64, wherein an AM dose and a PM dose are administered, and wherein the AM dose is larger or smaller than the PM dose.

117. The drug delivery regimen of claim 64, wherein an AM dose and a PM dose are administered, and wherein the AM dose has a higher or lower amount of a water-soluble active therapeutic substance(s) is present than that present in the PM dose.

118. The drug delivery regimen of claim 64, wherein an AM dose and a PM dose are administered, and wherein the AM dosage has a higher or lower amount of a non water-soluble drug present than that present in the PM dosage.

119. The drug delivery regimen of claim 64, wherein the dosage is adjusted for subsequent 24 hour periods of time.

120. The drug delivery regimen of claim 64, wherein the active therapeutic substance(s) is substituted for another active therapeutic substance(s).

121. The drug delivery regimen of claim 64, wherein two PM doses are administered, and wherein the first PM dose is administered immediately after dinner and the second PM dose is administered immediately prior to bedtime.

122. A.drug delivery regimen, which comprises:
at least two doses of an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, an anti-asthmatic, a hormone replacement agent, an anti-infective, an anti-diabetic, a vitamin, an herbal agent, a fatty acid, a mineral, an electrolyte and combinations thereof is administered during at least one 24 hour period of time to provide effective therapeutic levels of the active therapeutic substance or substances at a site or sites of action in an animal over said period, and wherein each dose is independently calculated according to known pharmacokinetic parameters of the active therapeutic substance(s) with variations to account for physiological anomalies which occur during said period to optimize levels of the active therapeutic substance(s) at the site or sites of action for maximum efficacy.

123. The drug delivery regimen of claim 122, wherein the anti-hypertensive agent is selected from the group consisting of a calcium channel blocker, an ACE inhibitor, an angiotensin II receptor antagonist, a beta-adrenoceptor antagonist, an alpha 1-adrenoceptor antagonists, an alpha 2-adrenoceptor antagonist, a diuretic and combinations thereof.

124. The drug delivery regimen of claim 123, wherein the calcium channel blocker is selected from the group consisting of nifedipine,. verapamil, nicardipine, diltiazem, isradipine, amlodipine, felodipine, nifedipine, bepridil and combinations thereof.

125. The drug delivery regimen of claim 123, wherein the ACE inhibitor is selected from the group consisting of quinapril, ramipril, captopril, benazepril, fosinopril, lisinopril, moexipril, enalapril and combinations thereof.

126. The drug delivery regimen of claim 123, wherein the angiotensin II receptor antagonist is losartan.

127. The drug delivery regimen of claim 123, wherein the beta adrenoceptor antagonist is selected from the group consisting of sotalol, timolol, esmolol, carteolol, propanolol, betaxolol, penbutolol, metoprolol, labetalol, acebutolol, atenolol, bisoprolol and combinations thereof.

128. The drug delivery regimen of claim 123, wherein the alpha 1-adrenoceptor antagonist is selected from the group consisting of doxazosin, phenoxybenzamine, guanethidine, guanadrel, terazosin, prazosin and combinations thereof.

129. The drug delivery regimen of claim 123, wherein the alpha 2-adrenoceptor agonist is selected from the group consisting of methyldopa, clonidine, guanfacine and combinations thereof.

130. The drug delivery regimen of claim 123, wherein the diuretic is selected from the group consisting of carbonic anhydrase inhibitors, loop diuretics, thiazides, potassium sparing diuretics and combinations thereof.

131. The drug delivery regimen of claim 122, wherein the osteoporotic agent is selected from the group consisting of alendronate, etidronate, pamidronate, clodronate, tiludronate, residronate, ibandronate and combinations thereof.

132. The drug delivery regimen of claim 122, wherein the GERD agent is selected from the group consisting of oral GI prokinetic agents, agents active against H. Pylori, proton pump inhibitors, H₂ histamine receptor antagonists, antacids and combinations thereof.

133. The drug delivery regimen of claim 132, wherein the oral GI prokinetic agent is selected from the group consisting of cisapride monohydrate, metoclopramide and combinations thereof.

134. The drug delivery regimen of claim 132, wherein the agent active against H. Pylori is selected from the group consisting of clarithromycin, tetracycline, amoxicillin, bismuth, metronidazole and combinations thereof.

135. The drug delivery regimen of claim 132, wherein the proton pump inhibitor is selected from the group consisting of omeprazole, lansoprazole and combinations thereof.

136. The drug delivery regimen of claim 132, wherein the H₂ histamine receptor antagonist is selected from the group consisting of cimetadine, famotidine, nizatidine, ranitidine, roxatidine and combinations thereof.

137. The drug delivery regimen of claim 122, wherein the anti-viral agent is selected from the group consisting of nucleoside analogs, protease inhibitors and combinations thereof.

138. The drug delivery regimen of claim 137, wherein the nucleoside analog is selected from the group consisting of zidovudine, azidothymidine, didanosine, zalcitabine, stavudine, lamivudine and combinations thereof.

139. The drug delivery regimen of claim 137, wherein the protease inhibitor is selected from the group consisting of saquinavir mesylate, ritonavir, indinavir and combinations thereof.

140. The drug delivery regimen of claim 122, wherein the anti-neoplastic agent is selected from the group consisting of cytoxic agents, anti-metabolites, platinum-containing compounds, antibiotic derivatives, fluoropyrimidines, nitrosoureas, vinca alkaloids, nitrogen mustard derivatives, adjuvant biological response modifiers and combinations thereof.

141. The drug delivery regimen of claim 140, wherein the cytoxic agent is selected from the group consisting of paclitaxel, cyclophosphamide, teniposide and combinations thereof.

142. The drug delivery regimen of claim 140, wherein the anti-metabolite is methotrexate.

143. The drug delivery regimen of claim 140, wherein the platinum-containing compound is selected from the group consisting of cisplatin (cis-diaminedichlororoplatinum), carboplatin, oxaliplatin and combinations thereof.

144. The drug delivery regimen of claim 140, wherein the antibiotic derivative is selected from the group consisting of adriamycin, bleomycin, dactinomycin, daunorubicin, doxorubicin, indarubicin, mytomycin and combinations thereof.

145. The drug delivery regimen of claim 140, wherein the fluoropyrimidine is selected from the group consisting of 5-FU (5-fluorouracil), FudR (5-fluoro-2′-deoxyuridine), Ara-C (arabinosylcytosine) and combinations thereof.

146. The drug delivery regimen of claim 140, wherein the nitrosourea is selected from the group consisting of BCNU (carmustine), streptozocin and combinations thereof.

147. The drug delivery regimen of claim 140, wherein the vinca alkaloid is selected from the group consisting of vinblastine, vincristine and combinations thereof.

148. The drug delivery regimen of claim 140, wherein the nitrogen mustard derivative is thiotepa.

149. The drug delivery regimen of claim 140, wherein the adjuvant biological response modifier is selected from the group consisting of alpha-interferon, TNF (tumor necrosis factor), EPO (erythropoietin), rhG-CSF (recombinant human granulocyte colony-stimulating factor), IL-1 (interleukin-1), IL-2 (interleukin-2), monoclonal antibodies to tumor and immunologic targets and combinations thereof.

150. The drug delivery regimen of claim 122, wherein the inhaled steroid is selected from the group consisting of beclomethasone dipropionate, budesonide, flunisolide, fluticasone propionate, mometasone furoate, triamcinolone acetonide and combinations thereof.

151. The drug delivery regimen of claim 122, wherein the lipid lowering agent is selected from the group consisting of nicotinic acid, HMG CoA reductase inhibitors, bile sequestration agents, fibric acid derivatives and combinations thereof.

152. The drug delivery regimen of claim 151, wherein the HMG CoA reductase inhibitor is selected from the group consisting of atorvastatin, cerivastatin, fluvastatin, lovastatin, pravastatin, simvastatin and combinations thereof.

153. The drug delivery regimen of claim 151, wherein the bile sequestration agent is selected from the group consisting of colestipol, cholestyramine and combinations thereof.

154. The drug delivery regimen of claim 151, wherein the fibric acid derivative is selected from the group consisting of clofibrate, gemfibrozil and combinations thereof.

155. The drug delivery regimen of claim 122, wherein the thrombolytic, anticoagulant, fibrinolytic agent is selected from the group consisting of heparin-like agents, clot buster agents, aspirin-like agents, platelet glycoprotein IIb, IIIa receptor antagonists and combinations thereof.

156. The drug delivery regimen of claim 154, wherein the heparin-like agent is selected from the group consisting of enoxaparin, dalteparin, refludan and combinations thereof.

157. The drug delivery regimen of claim 154, wherein the clot buster agent is selected from the group consisting of streptokinases, alteplase (TPA) and combinations thereof.

158. The drug delivery regimen of claim 155, wherein the aspirin-like agent is a thromboxane inhibitor.

159. The drug delivery regimen of claim 155, wherein the platelet glycoprotein IIb, IIIa receptor antagonist is selected from the group consisting of tirofiban, eptifibatide, abciximab and combinations thereof.

160. The drug delivery regimen of claim 122, wherein the vitamin(s) is selected from the group consisting of thiamine, niacinamide, pyridoxine, ascorbic acid, riboflavin, folic acid, vitamin A, vitamin E, vitamin D3, cyanocobalamin, biotin, pantothenic acid, derivatives thereof, and combinations thereof.

161. The drug delivery regimen of claim 122, wherein the herbal agent(s) is selected from the group consisting of black cohosh, licorice, false unicorn, siberian ginseng, sarsaparilla, squaw vine, blessed thistle, peppermint, spearmint, red raspberry, St. Johnswort, ginger, kola, hops, valerian, derivatives thereof and combinations thereof.

162. The drug delivery regimen of claim 122, wherein the fatty acid(s) is selected from the group consisting of linoleic acid, linolenic acid, docosahexaenoic acid, arachidonic acid, eicosahexaneoic acid, omega-3 fatty acid, omega-2 fatty acid, derivatives thereof and combinations thereof.

163. The drug delivery regimen of claim 122, wherein the mineral(s) is selected from the group consisting of copper, zinc, iodide, magnesium, chromium, molybdenum, sodium, calcium, iron, fluoride, phosphorus, manganese, potassium, boron, selenium, bioflavonoid, phosphate, derivatives thereof and combinations thereof.

164. The drug delivery regimen of claim 122, wherein the electrolyte(s) is selected from the group consisting of potassium, magnesium, sodium, calcium, derivatives thereof or combinations thereof.

165. A method of enhancing the therapeutic effect of an active therapeutic substance(s) selected from the group consisting of an anti-hypertensive agent, an osteoporotic agent, a GERD agent, an anti-viral agent, an anti-neoplastic agent, an inhaled steroid, a lipid lowering agent, a thrombolytic agent, an anticoagulant agent, a fibrinolytic agent, a hormone agent, an anti-arthritic agent, an antibiotic agent, an analgesic agent, a central nervous system agent, a psychotrophic agent, a vitamin, an herbal agent, a fatty acid, a mineral, an electrolyte and combinations thereof in an animal, which comprises:
(a) determining known pharmacokinetic parameters of the active therapeutic substance(s);
(b) determining a number of doses to be administered during a 24 hour period of time and determining a time at which each dose will be administered by considering both the animal's schedule and physiological anomalies during the 24 hour period; and
(c) independently calculating the amount of each dose in accordance with the equation
TD(t)=CD(t)+RD(t)
where t is the time at which the dose is to be administered, TD (therapeutic dose) is the therapeutically effective dose at time (t), CD (current dose) is the dose to be administered at time (t), RD (residual dose) is the amount of active therapeutic substance(s) remaining from the previous dose administration.

166. The method of claim 165, wherein the anti-hypertensive agent is selected from the group consisting of a calcium channel blocker, an ACE inhibitor, an angiotensin II receptor antagonist, a beta-adrenoceptor antagonist, an alpha 1-adrenoceptor antagonists, an alpha 2-adrenoceptor antagonist, a diuretic and combinations thereof.

167. The method of claim 166, wherein the calcium channel blocker is nifedipine, verapamil, nicardipine, diltiazem, isradipine, amlodipine, felodipine, nifedipine, bepridil or combinations thereof.

168. The method of claim 166, wherein the ACE inhibitor is quinapril, ramipril, captopril, benazepril, fosinopril, lisinopril, moexipril, enalapril or combinations thereof.

169. The method of claim 166, wherein the angiotensin II receptor antagonist is losartan.

170. The method of claim 166, wherein the beta adrenoceptor antagonist is sotalol, timolol, esmolol, carteolol, propanolol, betaxolol, penbutolol, metoprolol, labetalol, acebutolol, atenolol, bisoprolol or combinations thereof.

171. The method of claim 166, wherein the alpha 1-adrenoceptor antagonist is doxazosin, phenoxybenzamine, guanethidine, guanadrel, terazosin, prazosin or combinations thereof.

172. The method of claim 166, wherein the alpha 2-adrenoceptor agonist is methyldopa, clonidine, guanfacine or combinations thereof.

173. The method of claim 166, wherein the diuretic is selected from the group consisting of carbonic anhydrase inhibitors, loop diuretics, thiazides, potassium sparing diuretics or combinations thereof.

174. The method of claim 165, wherein the osteoporotic agent is alendronate, etidronate, pamidronate, clodronate, tiludronate, residronate, ibandronate or combinations thereof.

175. The method of claim 165, wherein the GERD agent is selected from the group consisting of oral GI prokinetic agents, agents active against H. Pylori, proton pump inhibitors, H₂ histamine receptor antagonists, antacids and combinations thereof.

176. The method of claim 175, wherein the oral GI prokinetic agent is cisapride monohydrate, metoclopramide or combinations thereof.

177. The method of claim 175, wherein the agent active against H. Pylori is clarithromycin, tetracycline, amoxicillin, bismuth, metronidazole or combinations thereof.

178. The method of claim 175, wherein the proton pump inhibitor is selected from the group consisting of omeprazole, lansoprazole and combinations thereof.

179. The method of claim 175, wherein the H₂ histamine receptor antagonist is cimetadine, famotidine, nizatidine, ranitidine, roxatidine and combinations thereof.

180. The method of claim 175, wherein the anti-viral agent is selected from the group consisting of nucleoside analogs, protease inhibitors and combinations thereof.

181. The method of claim 180, wherein the nucleoside analog is zidovudine, azidothymidine, didanosine, zalcitabine, stavudine, lamivudine or combinations thereof.

182. The method of claim 180, wherein the protease inhibitor is selected from the group consisting of saquinavir mesylate, ritonavir, indinavir and combinations thereof.

183. The method of claim 165, wherein the anti-neoplastic agent is selected from the group consisting of cytoxic agents, anti-metabolites, platinum-containing compounds, antibiotic derivatives, fluoropyrimidines, nitrosoureas, vinca alkaloids, nitrogen mustard derivatives, adjuvant biological response modifiers and combinations thereof.

184. The method of claim 183, wherein the cytoxic agent is selected from the group consisting of placlitaxel, cyclophosphamide, teniposide and combinations thereof.

185. The method of claim 183, wherein the anti-metabolite is methotrexate.

186. The method of claim 183, wherein the platinum-containing compound is selected from the group consisting of cisplatin (cis-diaminedichlororoplatinum), carboplatin, oxaliplatin and combinations thereof.

187. The method of claim 183, wherein the antibiotic derivative is adriamycin, bleomycin, dactinomycin, daunorubicin, doxorubicin, indarubicin, mytomycin or combinations thereof.

188. The method of claim 183, wherein the fluoropyrimidine is 5-FU (5-fluorouracil), FudR (5-fluoro-2′-deoxyuridine), Ara-C (arabinosylcytosine) or combinations thereof.

189. The method of claim 183, wherein the nitrosourea is BCNU (carmustine), streptozocin or combinations thereof.

190. The method of claim 183, wherein the vinca alkaloid is vinblastine, vincristine or combinations thereof.

191. The method of claim 183, wherein the nitrogen mustard derivative is thiotepa.

192. The method of claim 183, wherein the adjuvant biological response modifier is selected from the group consisting of alpha-interferon, TNF (tumor necrosis factor), EPO (erythropoietin), rhG-CSF (recombinant human granulocyte colony-stimulating factor), IL-1 (interleukin-1), IL-2 (interleukin-2), monoclonal antibodies to tumor and immunologic targets and combinations thereof.

193. The method of claim 165, wherein the inhaled steroid is beclomethasone dipropionate, budesonide, flunisolide, fluticasone propionate, mometasone furoate, triamcinolone acetonide or combinations thereof.

194. The method of claim 165, wherein the lipid lowering agent is selected from the group consisting of nicotinic acid, HMG CoA reductase inhibitors, bile sequestration agents, fibric acid derivatives and combinations thereof.

195. The method of claim 194, wherein the HMG CoA reductase inhibitor is atorvastatin, cerivastatin, fluvastatin, lovastatin, pravastatin, simvastatin or combinations thereof.

196. The method of claim 194, wherein the bile sequestration agent is colestipol, cholestyramine or combinations thereof.

197. The method of claim 194, wherein the fibric acid derivative is clofibrate, gemfibrozil or combinations thereof.

198. The method of claim 165, wherein the thrombolytic, anticoagulant, fibrinolytic agent is selected from the group consisting of heparin-like agents, clot buster agents, aspirin-like agents, platelet glycoprotein IIb, IIIa receptor antagonists and combinations thereof.

199. The method of claim 198, wherein the heparin-like agent is enoxaparin, dalteparin, refludan or combinations thereof.

200. The method of claim 198, wherein the clot buster agent is selected from the group consisting of streptokinases, alteplase (TPA) and combinations thereof.

201. The method of claim 198, wherein the aspirin-like agent is a thromboxane inhibitor.

202. The method of claim 198, wherein the platelet glycoprotein IIb, IIIa receptor antagonist is tirofiban, eptifibatide, abciximab or combinations thereof.

203. The method of claim 165, wherein the vitamin is thiamine, niacinamide, pyridoxine, ascorbic acid, riboflavin, folic acid, vitamin A, vitamin E, vitamin D3, cyanocobalamin, biotin, pantothenic acid, derivatives thereof or combinations thereof.

204. The method of claim 165, wherein the herbal agent is black cohosh, licorice, false unicorn, siberian ginseng, sarsaparilla, squaw vine, blessed thistle, peppermint, spearmint, red raspberry, St. Johnswort, ginger, kola, hops, valerian, derivatives thereof or combinations thereof.

205. The method of claim 165, wherein the fatty acid is selected from the group consisting of linoleic acid, linolenic acid, docosahexaenoic acid, arachidonic acid, eicosahexaneoic acid, omega-3 fatty acid, omega-2 fatty acid, derivatives thereof and combinations thereof.

206. The method of claim 165, wherein the mineral is selected from the group consisting of copper, zinc, iodide, magnesium, chromium, molybdenum, sodium, calcium, iron, fluoride, phosphorus, manganese, potassium, boron, selenium, bioflavonoid, phosphate, derivatives thereof and combinations thereof.

207. The method of claim 165, wherein the electrolyte is selected from the group consisting of potassium, magnesium, sodium, calcium, derivatives thereof and combinations thereof.

208. The method of claim 165, wherein the active therapeutic substance(s) is administered at least twice and may be administered as immediate release, sustained release, controlled release, delayed release, timed release, extended release or any combination thereof.

209. The method of claim 165, wherein the active therapeutic substance(s) is administered by pulsatile delivery of the active therapeutic substance(s).

210. The method of claim 165, wherein the active therapeutic substance(s) is administered in a dosage form independently selected from the group consisting of Examples of such dosage forms include, without limitation, chewable tablets, quick dissolve tablets, effervescent tablets, reconstitutable powders, elixirs, liquids, solutions, suspensions, emulsions, tablets, multi-layer tablets, bi-layer tablets, capsules, soft gelatin capsules, hard gelatin capsules, caplets, lozenges, chewable lozenges, beads, powders, granules, particles, microparticles, dispersible granules, cachets, douches, suppositories, creams, topicals, inhalants, aerosol inhalants, patches, particle inhalants, implants, depot implants, ingestibles, injectables, infusions, health bars, confections, animal feeds, cereals, cereal coatings, foods, nutritive foods, functional foods, by a vaporizer and combinations thereof.

211. The method of claim 165, wherein the active therapeutic substance(s) is administered in a dosage form independently selected from the group consisting of a tablet, multi-layer tablet, capsule and caplet.

212. The method of claim 165, wherein the active therapeutic substance(s) is administered in uneven doses.

213. The method of claim 165, wherein the active therapeutic substance(s) is administered at uneven time intervals over the course of the 24 hour period.

214. The method of claim 165, wherein an AM dose and a PM dose are administered, and wherein the AM dose is larger or smaller than the PM dose.

215. The method of claim 165, wherein an AM dose and a PM dose are administered, and wherein the AM dose has a higher or lower amount of a water-soluble active therapeutic substance(s) present than that present in the PM dose.

216. The method of claim 165, wherein an AM dose and a PM dose are administered, and wherein the AM dosage has a higher or lower amount of a non water-soluble drug present than that present in the PM dosage.

217. The method of claim 165, wherein the dosage is adjusted for subsequent 24 hour periods of time.

218. The method of claim 165, wherein the active therapeutic substance(s) is substituted for another active therapeutic substance(s).

219. The method of claim 165, wherein two PM doses are administered, and wherein the first PM dose is administered immediately after dinner and the second PM dose is administered immediately prior to bedtime.

220. A method for maximizing therapeutic effectiveness of an antihypertensive agent, which comprises:
administering a first dose of the antihypertensive agent at a first preselected time during a twenty four hour period;
administering a second dose of the antihypertensive agent at a second preselected time during the twenty four hour period;
wherein said first dose is about 30% of the total amount of the antihypertensive agent to be administered during the twenty four hour period and the second dose is about 70% of the total amount of the antihypertensive agent to be administered during the twenty four hour period;
and wherein said first preselected time is about 6-8 am and the second preselected time is about 6-8 pm.

221. The method of claim 220, wherein the anti-hypertensive agent is selected from the group consisting of a calcium channel blocker, an ACE inhibitor, an angiotensin II receptor antagonist, a beta-adrenoceptor antagonist, an alpha 1-adrenoceptor antagonists, an alpha 2-adrenoceptor antagonist, a diuretic and combinations thereof.

222. The method of claim 221, wherein the calcium channel blocker is selected from the group consisting of nifedipine, verapamil, nicardipine, diltiazem, isradipine, amlodipine, felodipine, nifedipine, bepridil and combinations thereof.

223. The method of claim 221, wherein the ACE inhibitor is selected from the group consisting of quinapril, ramipril, captopril, benazepril, fosinopril, lisinopril, moexipril, enalapril and combinations thereof.

224. The method of claim 221, wherein the angiotensin II receptor antagonist is losartan.

225. The method of claim 221, wherein the beta adrenoceptor antagonist is selected from the group consisting of sotalol, timolol, esmolol, carteolol, propanolol, betaxolol, penbutolol, metoprolol, labetalol, acebutolol, atenolol, bisoprolol and combinations thereof.

226. The method of claim 221, wherein the alpha 1-adrenoceptor antagonist is selected from the group consisting of doxazosin, phenoxybenzamine, guanethidine, guanadrel, terazosin, prazosin and combinations thereof.

227. The method of claim 221, wherein the alpha 2-adrenoceptor agonist is selected from the group consisting of methyldopa, clonidine, guanfacine and combinations thereof.

228. The method of claim 221, wherein the diuretic is selected from the group consisting of carbonic anhydrase inhibitors, loop diuretics, thiazides, potassium sparing diuretics and combinations thereof.

229. A method for maximizing therapeutic effectiveness of an osteoporotic agent, which comprises:
administering a first dose of the osteoporotic agent at a first preselected time during a twenty four hour period of time to an animal;
administering a second dose of the osteoporotic agent at a second preselected time during the twenty four hour period of time to the animal;
wherein said first dose is about 25% to about 35% of the total amount of the osteoporotic agent to be administered during the twenty four hour period of time and the second dose is about 65% to about 75% of the total amount of the osteoporotic agent to be administered during the twenty four hour period of time;
and wherein said first preselected time is the period between the animal's awakening until just after the animal's morning meal and the second preselected time is the period between the animal's evening meal and the animal's bedtime.

230. The method of claim 229, wherein the osteoporotic agent is selected from the group consisting of alendronate, etidronate, pamidronate, clodronate, tiludronate, residronate, ibandronate and combinations thereof.

231. A method for maximizing therapeutic effectiveness of AZT, which comprises:
administering a first dose of AZT at a first preselected time during a twenty four hour period of time to an animal;
administering a second dose of AZT at a second preselected time during the twenty four hour period of time to the animal;
administering a third dose of AZT at a third preselected time during the twenty four hour period of time to the animal;
wherein said first dose and the third dose are equal and at least 70% of the second dose;
and wherein said first preselected time is from 6 am to 9 am, the second preselected time is from 3 pm to 6 pm and the third preselected time is from 9 pm to 12 pm.

232. A pharmaceutical composition for optimizing therapeutic activity, which comprises:
a first active therapeutic substance(s) selected from the group consisting of water-soluble vitamins, water-soluble minerals and water-soluble electrolytes; and
a second active therapeutic substance(s) selected from the group consisting of non water-soluble vitamins, non water-soluble minerals and fatty acids;
wherein the ratio of the first active therapeutic substance(s) to the second active therapeutic substance(s) is independently tailored to optimize levels of the respective active therapeutic substances at a site or sites of action in an animal for maximum efficacy, and wherein said weight ratio is determined according to the time at which said composition is to be administered with a suitable pharmaceutical carrier.

233. The pharmaceutical composition of claim 232, wherein the water-soluble vitamin is selected from the group consisting of vitamin B₁, vitamin B₂, vitamin B₃, biotin, pantothenic acid, vitamin B₆, folate, vitamin B₁₂, vitamin C, derivatives thereof and combinations thereof.

234. The pharmaceutical composition of claim 232, wherein the water-soluble mineral is selected from the group consisting of sodium, potassium, calcium, phosphorus, magnesium, sulfur, ferrous iron, zinc, iodide, copper, fluoride, derivatives thereof and combinations thereof.

235. The pharmaceutical composition of claim 232, wherein the water-soluble electrolyte is selected from the group consisting of sodium, potassium, calcium, magnesium, derivatives thereof and combinations thereof.

236. The pharmaceutical composition of claim 232, wherein the non water-soluble vitamin is selected from the group consisting of vitamin A, vitamin D, vitamin E, vitamin K, derivatives thereof and combinations thereof.

237. The pharmaceutical composition of claim 232, wherein the non water-soluble mineral is selected from the group consisting of chromium, ferric iron, molybdenum, selenium, derivatives thereof and combinations thereof.

238. The pharmaceutical composition of claim 232, wherein the fatty acid is selected from the group consisting of linoleic acid, linolenic acid, arachidonic acid, eicopentaenoic acid, docosahexaenoic acid, omega-2 fatty acid, omega-3 fatty acid, derivatives thereof and combinations thereof.
